(19) United States
(12) Patent Application Publication
(10) Pub. No.: US 2017/0239391 A1
(43) Pub. Date: Aug. 24, 2017

(54) SYSTEMS AND METHODS FOR PROVIDING OXYGEN TO TRANSPLANTED CELLS

(71) Applicant: Beta-O2 Technologies Ltd., Rosh-Haayin (IL)

(72) Inventors: Avi Rotem, Petach-Tikva (IL); Yuval Avni, Petach-Tikva (IL)

(73) Assignee: Beta-O2 Technologies Ltd., Rosh-Haayin (IL)

(21) Appl. No.: 15/426,535

(22) Filed: Feb. 7, 2017

Related U.S. Application Data
(60) Provisional application No. 62/292,623, filed on Feb. 8, 2016.

(51) Int. Cl. A61L 27/38; A61L 27/52; A61L 27/34; A61F 2/02; A61L 27/16; A61M 1/16; A61M 39/02; A61L 27/56; A61K 35/39

(52) U.S. Cl. CPC A61L 27/3804; A61L 27/56; A61L 27/52; A61L 27/34; A61K 35/39; A61L 27/16; A61M 1/1678; A61M 39/0208; A61F 2/022; A61L 2300/64; A61K 2035/126

(57) ABSTRACT

A device containing transplanted tissue includes a housing, having a chamber configured for insertion into a body of a subject and protecting the transplanted tissue from the subject's immune system. The housing includes an oxygen supply container, a hydrogel layer, a port, and an access port. The oxygen supply container has a chamber defined by top and bottom surfaces and sides, disposed within the chamber of the housing. The top surface and the bottom surface of the oxygen supply container include a gas-permeable membrane. The hydrogel layer has inner and outer surfaces. The inner surface of the hydrogel layer contacts the top surface of the oxygen supply container or the bottom surface of the oxygen supply container. The port is configured to deliver oxygen to the oxygen supply container. The access port is configured to receive an exogenous supply of gas and is fluidly connected to the port.

### CROSS REFERENCE TO RELATED APPLICATIONS

This application claims priority to U.S. Provisional Patent Application Ser. No. 62/292,623, filed on Feb. 8, 2016, the entire contents of which are hereby incorporated by reference in their entirety.

### FIELD OF THE INVENTION

The field of invention relates to medical devices, cell therapies and medical devices containing cells. In particular, the present invention provides an apparatus for promoting the survival and function of transplanted cells.

### BACKGROUND OF THE INVENTION

Organ transplantation is often not a viable treatment hormone disorders, such as, for example, diabetes. Frequently, the transplanted tissue, or the transplanted cells are in short supply, and can be rejected by the recipient. Isolated tissue or cells may be transplanted in the body after being treated to prevent rejection, such as, for example, by immunosuppression, radiation or encapsulation.

Transplants may also fail due to ischemic conditions generated by insufficient oxygen supply to the transplant. For example, by way of illustration, donor islets are isolated from pancreatic tissue by enzymatic and mechanical processing, which disrupts their blood supply, thus limiting the diffusion of oxygen to the islets.

Oxygen is vital for the physiological processes, viability, and functionality of the transplanted cells. An insufficient supply of oxygen to the implanted cells, often leads to loss of functionality, and/or death of the transplanted cells.

For example, by way of illustration using islets as an example of transplanted functional cells, initially, transplanted islets receive oxygen from the recipient's blood supply by diffusion. In some cases, vascular structures can eventually form around the transplanted islets with the help of, for example, angiogenic factors, e.g., VEGF and bFGF, which may increase the efficiency or rate of oxygen diffusion. In order to protect the transplanted islets from the immune system, the transplanted islets are often protected by encapsulation, isolating the transplanted islets from the recipient's immune system.

However, the diffusion of oxygen to the transplanted cells can be reduced if the transplanted cells are encapsulated. Additionally, the demand of the transplanted cells can be affected by the amount of cells transplanted. For example, the demand for oxygen of highly dense implanted cells may be higher than the diffusion capacity, resulting in lake of oxygen to the implanted cells. Moreover, highly metabolically active cells, such as, for example, insulin producing cells frequently require greater amounts of oxygen to be supplied to the transplanted tissue.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A, 1B, and 1C show an embodiment of the device of the present invention, showing the implantable device. FIG. 1A shows a schematic cross section of an embodiment of a device. FIG. 1B shows a schematic of an embodiment of the device of the present invention. FIG. 1C shows microphotographs of top surfaces of three devices with different islet densities (2,400 IEQ/cm², 3,600 IEQ/cm²; 4,800 IEQ/cm², respectively). Individual islets encapsulated in the at least one hydrogel layer are visible beneath the top metal gird. Bar is 1.8 mm.

FIG. 2 shows a representation of a cross-section of a conical cell utilized for O₂ measurements on devices according to some embodiments of the present invention. Drawing is not to scale. Dimensions are in mm.

FIG. 3 shows a schematic drawing of system to measure the oxygen profile within the at least one hydrogel layer containing the transplanted tissue. In the embodiment shown, islets are immobilized in the at least one hydrogel layer within the device, but without the Biopore membrane and the top metal grid. The oxygen supply container is purged with gas mixtures having various O₂ levels (the outlet port is not shown), and an O₂ electrode is gradually inserted into the area containing the transplanted tissue. The thickness of the at least one hydrogel layer is not in proportion in order to show the O₂ electrode mechanism.

FIGS. 4A and 4B show the oxygen profile within the at least one hydrogel layer containing the transplanted tissue in a device according to some embodiments of the present invention. 2,400 IEQ with OCR of 3.5 pmol/IEQ/min were immobilized in a 600-μm thick hydrogel layer, at a density of 4,800 IEQ/cm². The O₂ electrode was inserted at the interface or between the immobilized islets and medium and moved sequentially at increments of 100 μm down to the interface of the gas permeable membrane. FIG. 4A: Representative raw data. FIG. 4B: O₂ partial pressure profile calculated from the data in FIG. 4A.

FIG. 5 A shows the ability of devices according to some embodiments of the present invention to lower blood glucose, when transplanted into diabetic rats. The panels show data obtained from devices containing various densities of donor islets, indicated in the top right of the panels, oxygenated with different oxygen concentrations (See Table 2). The arrows indicate when devices were removed. The traces are the average observed blood glucose levels. FIG. 5 B shows the results of an intravenous glucose tolerance test (IVGTT) over about 180 minutes, performed 6 weeks post implantation. Normal non-diabetic rat (full diamond); Diabetic animals implanted with the device containing islets at average densities of: 1,000 IEQ/cm²; 2,400 IEQ/cm²; 3,600 IEQ/cm² and 4,800 IEQ/cm² (full triangle).

FIG. 6 shows the average observed oxygen consumption rate of 2,400 IEQ within devices according to some embodiments of the present invention, at the densities indicated. The dark bars show the rate of oxygen consumption prior to implantation. The light bars show the rate of oxygen consumption after the devices removed, following implantation for a minimum of 90 days.

FIGS. 7 A and B shows naïve micrographs of the dense vascular structures within the islets before isolation of the islets.

FIG. 8 A shows a micrograph of a cross-section of the at least one hydrogel layer containing the transplanted tissue in a device according to some embodiments of the present invention. The arrow indicates the direction at which oxygen diffuses through the at least one hydrogel layer. FIG. 8 B shows theoretical oxygen gradients (dashed lines) through the at least one hydrogel layer, illustrating the maximum dissolved oxygen and minimum dissolved oxygen concentration from the inner surface of the at least one hydrogel layer (801) to the outer surface of the at least one hydrogel layer (802). The different dashed lines indicate theoretical oxygen gradients in different islet densities. The outer surface of the at least one hydrogel layer (802) is adjacent to the recipient's blood supply.

FIG. 9 shows a photograph of a rat with a device according to an embodiment of the present invention implanted subcutaneously.

FIG. 10 shows the ability of devices according to some embodiments of the present invention to lower blood glucose, when transplanted into diabetic rats. FIG. 10 A shows the blood glucose prior to implantation (−10 to 0), and following implantation of a device according to some embodiments of the present invention, but without added oxygen. FIG. 10 B shows the blood glucose prior to implantation (−10 to 0), and following implantation of a device according to some embodiments of the present invention, where oxygen was supplied to the device according to the methods described in some embodiments of the present invention. The arrow indicates when oxygen was replaced with nitrogen.

FIG. 11 shows a micrograph of a fibrotic pocket surrounding a device removed from a rat after being implanted for a period of 140 days.

FIG. 12 shows results from another IVGTT, showing blood glucose levels observed from rats implanted with devices according to some embodiments of the present invention, containing isogeneic (triangle) or allogeneic (circle) islets. Blood glucose levels observed from non-diabetic animals (square), and non-treated diabetic animals (diamond) are also shown.

FIG. 13 shows an embodiment of a device of the present invention. In the embodiment shown, the device is a large device for large animals, such as pigs or humans.

FIG. 14 shows a cross section of the device shown in FIG. 13.

FIG. 15 A shows the average body mass (squares) and blood glucose levels (circles) from 4 pigs implanted with devices according to some embodiments of the present invention, containing rat islets. FIG. 15 B shows insulin staining in islets that were retrieved from the device after 89 days of implantation.

FIG. 16 A shows validation of PCR reactions using the primers indicated, from tissue removed from devices according to some embodiments of the present invention, containing rat islets that were implanted into pigs. FIG. 16 B shows a representation of the technique used to remove the transplanted tissue sample. FIG. 16 C shows the results of PCR reactions using the primers indicated, from tissue removed from devices according to some embodiments of the present invention, containing rat islets that were implanted into pigs.

FIG. 17 A shows the rate of insulin diffusion across a hydrophilized Teflon membrane impregnated with the High manuronic alginate hydrogel (HM-DM), utilized in a device according to some embodiments of the present invention (squares), and a non-impregnated Teflon membrane control (diamonds).

FIG. 17 B shows the diffusion of IgG across a hydrophilic Teflon membrane impregnated with the High manuronic alginate, utilized in a device according to some embodiments of the present invention (squares), and a non-impregnated Teflon membrane control (diamonds).

FIG. 18 A shows a representation of an experimental system to test the ability of a device according to some embodiments of the present invention to block the transfer of viruses between the transplanted tissue and the recipient. FIG. 18 B shows the passage of virus across a Teflon membrane impregnated with the hydrogel HM DM, utilized in a device according to some embodiments of the present invention (diamonds, on the bottom of the figure), and a non-impregnated Teflon membrane control (circles).

FIG. 19 A shows implantation sites on a human subject for a device according to some embodiments of the present invention. FIG. 19 B shows the implantation of a device according to some embodiments of the present invention into a human subject. FIG. 19 C shows another view of the implantation of a device according to some embodiments of the present invention into a human subject. FIG. 19 D shows another view of the implantation of a device according to some embodiments of the present invention into a human subject.

FIG. 20 A shows the blood glucose levels a diabetic human patient receiving insulin injections, prior to being implanted with a device according to some embodiments of the present invention. The individual traces show blood glucose levels for a single 24 hour period. FIG. 20 B shows the blood glucose levels a diabetic human patient implanted with a device according to some embodiments of the present invention. Data was obtained 1 month post-implantation. The individual traces show blood glucose levels for a single 24 hour period.

FIG. 21 A shows fructosamine levels in a human subject implanted with a device according to some embodiments of the present invention, pre- and post-implantation. FIG. 21 B shows hemoglobin A1c levels in a human subject implanted with a device according to some embodiments of the present invention, pre- and post-implantation. FIG. 21 C shows glucose-stimulated c-peptide secretion from the implanted device according to some embodiments of the present invention, 3, 6, and 9 months post implantation.

FIG. 22 shows glucose-stimulated insulin, pro-insulin, and c-peptide secretion from the implanted device according to some embodiments of the present invention at the times indicated.

FIG. 23 A shows a micrograph of a device according to some embodiments of the present invention, after removal from a human subject, after being implanted for 10 months. FIG. 23 B shows a micrograph of islets stained with dithizone, in a device according to some embodiments of the present invention, after removal from a human subject, after being implanted for 10 months.

FIG. 24 A shows glucose-stimulated insulin secretion from islets in a device according to some embodiments of the present invention, after removal from a human subject, after being implanted for 10 months. FIG. 24 B shows glucose-stimulated c-peptide production from islets in a device according to some embodiments of the present invention, after removal from a human subject, after being implanted for 10 months.

FIG. 25 shows a schematic illustration of a cross-section of a cylindrical or ellipsoidal device according to some embodiments of the present invention.

FIG. 26 shows a schematic illustration of a method to manufacture a composite membrane according to some embodiments of the present invention.

FIG. 27 shows a schematic illustration of a composite membrane produced according to the method shown in FIG. 26.

FIG. 28 shows a schematic illustration of a method to manufacture a device according to some embodiments of the present invention.

FIG. 29 shows a schematic illustration of a method to manufacture a device according to some embodiments of the present invention.

FIG. 30 shows a schematic illustration of a cross-section of a device according to some embodiments of the present invention.

FIGS. 31A and 31B show human islets in a device according to some embodiments of the present invention. FIG. 31A shows a micrograph of human islets in a device according to some embodiments of the present invention prior to implantation in a rat. FIG. 31B shows a micrograph of human islets in a device according to some embodiments of the present invention in a device removed from a rat after being implanted for one month.

FIG. 32 A shows basal and ACTH-stimulated plasma cortisol levels in adrenalectomized rats (ADX), adrenalectomized rats implanted with a device according to some embodiments of the present invention containing bovine adrenal cells (DEVICE), and adrenalectomized rats implanted with alginate hydrogels containing bovine adrenal cells (SLABS). FIG. 32 B shows the viability of bovine adrenal cells in a device according to some embodiments of the present invention. Data was obtained following 20 days of implantation.

FIG. 33 shows C-peptide levels in diabetic rats and stage 4 human stem cell implanted with a device according to some embodiments of the present invention.

### SUMMARY OF THE INVENTION

In one embodiment, the present invention provides a device containing transplanted cells, comprising:
a housing, having a chamber, defined by a top, a bottom surface, and sides, configured for insertion into a body of a subject, comprising:
   a. an oxygen supply container, having a chamber, defined by a top surface, a bottom surface, and sides, disposed within the chamber of the housing, wherein the top surface and the bottom surface of the oxygen supply container comprise at least one gas-permeable membrane,
   b. at least one hydrogel layer, having an inner surface, and an outer surface, wherein the inner surface of the at least one hydrogel layer contacts at least one surface selected from the group consisting of: the top surface of the oxygen supply container, and the bottom surface of the oxygen supply container, wherein the at least one hydrogel layer contains the transplanted cells;
   c. at least one port, configured to deliver oxygen to the oxygen supply container, wherein the at least one port is fluidly connected to the chamber of the oxygen supply container; and
   d. at least one access port, configured to receive an exogenous supply of gas, fluidly connected to the at least one port,
wherein the device is configured to promote the survival and/or function of the transplanted cells;
wherein the oxygen supply container is further configured to supply oxygen to provide a minimum pO₂ of between a value of 50-600 mm Hg for at least 24 hours, and
wherein the oxygen supply container is further configured to be periodically replenished with oxygen.

In one embodiment, the at least one hydrogel layer comprises guluronic acid alginate.

In one embodiment, the transplanted cells are selected from the group consisting of islets of Langerhans, stem cells, adrenal cells, insulin secreting cells, beta cells, stem cell-derived insulin producing cells, stem cell-derived beta cells, stem cell-derived alpha cells and genetically modified cells.

In one embodiment, the transplanted cells are human.

In one embodiment, the transplanted cells are allogeneic. In one embodiment, the transplanted cells are xenogeneic. In one embodiment, the transplanted cells are isogeneic. In one embodiment, the transplanted cells are autologous.

In one embodiment, the device protects the transplanted cells from the subject's immune system.

In one embodiment, the outer surface of the at least one hydrogel layer comprises an immune protection membrane.

In one embodiment, the immune protection membrane comprises polytetrafluoroethylene or collagen. In one embodiment, the at least one gas-permeable membrane comprises silicone rubber-teflon.

In one embodiment, the device is implanted into the body of the subject at a location selected from the group consisting of: a subcutaneous location, an intramuscular location, an intraperitoneal location, a pre-peritoneal location, and an omental location.

In one embodiment, the oxygen delivered to the chamber of the oxygen supply container has a concentration between 21% and 95%.

In one embodiment, the oxygen is delivered to the chamber of the oxygen supply container at an initial partial pressure of between 200 and 950 mmHg.

In one embodiment, the transplanted cells contained within the at least hydrogel layer has a density of a value between 1,000,000 cells/cm² and 100,000,000 cells/cm².

In one embodiment, the transplanted cells contained within the at least one hydrogel layer has a density of a value between 1,000 IEQ/cm² and 15,000 IEQ/cm².

In one embodiment, the least hydrogel layer has a uniform thickness between 100 and 800 micrometers.

In one embodiment, the at least one access port is implanted remotely from the apparatus. In one embodiment, the at least one access port is implanted into the body of the subject at a location selected from the group consisting of: a subcutaneous location, an intramuscular location, an intraperitoneal location, a pre-peritoneal location, a pre-peritoneal location, and an omental location.

In one embodiment, oxygen passes from the chamber of the oxygen supply container to the transplanted cells contained within the hydrogel layer through the at least one gas-permeable membrane of the oxygen supply container.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, “islet equivalents” or “IEQ” refers to the volume of a spherical islet with a diameter of 150 microns (μm). Each islet contains between 1,000 cells to 4,000 cells, which includes transplanted cells (e.g., but not limited to, beta cells).

As used herein, “IEQ/cm³” refers to the density of the islets. In clinical practice, densities can range from approximately 1,000-10,000 IEQ/cm². Since each islet can contain between 1,000-4,000 transplanted cells, as a non-limiting example, 1,000 IEQ/cm² can contain 3,000,000-4,000,000 transplanted cells.

As used herein, “functionality” refers to the biological activity of the transplanted tissue, such as, for example, glucose-responsive insulin secretion.

As used herein, “allogeneic” refers to different gene constitutions within the same species; thus, antigenically distinct.

As used herein, “xenogeneic” refers to heterologous, with respect to tissue grafts, e.g., when donor and recipient belong to different species.

As used herein, “isogeneic” refers to identical gene constitutions; thus, antigenically identical.

As used herein, “autologous” refers to a graft in which the donor and the recipient are the same individual.

Without being intended to be limited by any particular theory, oxygen is vital for the physiological processes and functionality of the transplanted cells. An insufficient supply of oxygen to the transplanted cells, often leads to cell loss of functionality or cell death. Thus, oxygen provision is a vital component in sustaining the viability and functionality of transplanted cells. In some embodiments, the device of the present invention is configured to supply oxygen to transplanted cells contained within the device, to maintain viability, and/or functionality of the transplanted cells.

In some embodiments, the present invention provides a device containing transplanted cells, comprising:
a housing, having a chamber, defined by a top, a bottom surface, and sides, configured for insertion into a body of a subject, comprising:
   a. an oxygen supply container, having a chamber, defined by a top surface, a bottom surface, and sides, disposed within the chamber of the housing, wherein the top surface and the bottom surface of the oxygen supply container comprise at least one gas-permeable membrane,
   b. at least one hydrogel layer, having an inner surface, and an outer surface, wherein the inner surface of the at least one hydrogel layer contacts at least one surface selected from the group consisting of: the top surface of the oxygen supply container, and the bottom surface of the oxygen supply container, wherein the at least one hydrogel layer contains the transplanted cells;
   c. at least one port, configured to deliver oxygen to the oxygen supply container, wherein the at least one port is fluidly connected to the chamber of the oxygen supply container; and
   d. at least one access port, configured to receive an exogenous supply of gas, fluidly connected to the at least one port,
wherein the device is configured to promote the survival and/or function of the transplanted cells;
wherein the oxygen supply container is further configured to supply oxygen to provide a minimum pO2 of between a value of 20-600 mm Hg for at least 24 hours, and
wherein the oxygen supply container is further configured to be periodically replenished with oxygen.

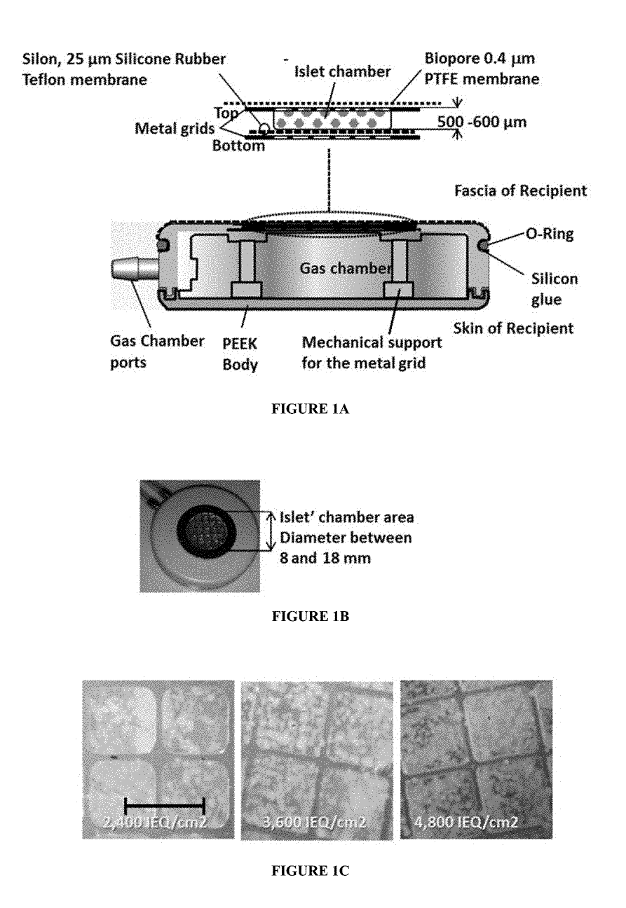

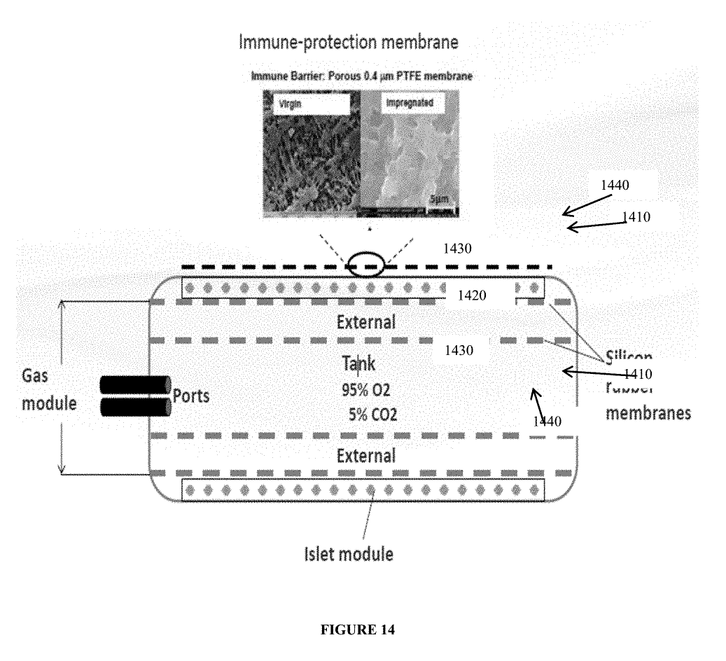

In some embodiments, the device of the present invention is the device disclosed in FIG. 1. Alternatively, the device of the present invention is the device disclosed in FIG. 13. Alternatively, the device of the present invention is the device disclosed in FIG. 14.

Referring to FIG. 13, the device has a diameter of 68 mm and a thickness of 17 mm.

Referring to FIG. 14, oxygen is replenished every 24 hours into the oxygen supply container via the ports, where the gas includes 5% CO₂ and 95% O2 at a pressure of 0.4 atm above ambient O₂ atm (Tank 1420). 1410 shows an area within the device (adjacent to the external regions) which houses transplanted cells and, after 24 hours has elapsed since gas was replenished into the oxygen supply container, the O₂ level is measured at about approximately 305 mg Hg at a density of 4,800 IEQ/cm2. In some embodiments, 305 mg Hg is the minimal level of oxygen required to satisfy the oxygen needs of the transplanted cells housed in the device. The oxygen supply container (Tank 1420) permits the diffusion of oxygen into the external regions (1430). The far end of the at least one hydrogel layer containing transplanted cells (1440) has an O₂ level of between 30-65 mg Hg after 24 hours.

In some embodiments, the device of the present invention comprises an external disc-shaped housing made of clinical grade polyether ether ketone. Alternatively, in some embodiments, the housing is formed from the material described in U.S. Pat. No. 8,821,431 B2. Alternatively, in some embodiments, the housing is formed from the material described in U.S. Pat. No. 8,784,389 B2. Alternatively, in some embodiments, the housing is formed from the material described in U.S. Pat. No. 8,444,630 B2. Alternatively, in some embodiments, the housing is formed from the material described in U.S. Pat. No. 8,012,500 B2. Alternatively, in some embodiments, the housing is formed from the material described in U.S. Patent Application Publication No. 20110300191 A1. Alternatively, in some embodiments, the housing is formed from the material described in U.S. Patent Application Publication No. 20150273200 A1.

In some embodiments, the device of the present invention is assembled according to the methods described in U.S. Pat. No. 8,821,431 B2. Alternatively, in some embodiments, the device of the present invention is assembled according to the methods described in U.S. Pat. No. 8,784,389 B2. Alternatively, in some embodiments, the device of the present invention is assembled according to the methods described in U.S. Pat. No. 8,444,630 B2. Alternatively, in some embodiments, the device of the present invention is assembled according to the methods described in U.S. Pat. No. 8,012,500 B2. Alternatively, in some embodiments, the device of the present invention is assembled according to the methods described in U.S. Patent Application Publication No. 20110300191 A1. Alternatively, in some embodiments, the device of the present invention is assembled according to the methods described in U.S. Patent Application Publication No. 20150273200 A1. Alternatively, the device of the present invention is assembled according to the methods described in Example 1 below.

In some embodiments, the device protects the transplanted cells from the subject's immune system.

In some embodiments, the outer surface of the at least one hydrogel layer comprises an immune protection membrane. In some embodiments, the transplanted cells are protected from the subject's immune system via the immune protection membrane.

In some embodiments, the immune protection membrane comprises porous polytetrafluoroethylene or collagen.

In some embodiments, the immune protection membrane comprises the immune protection membrane disclosed in U.S. Patent Application Publication No. 20110300191 A1.

In some embodiments, the immune protection membrane comprises a composite membrane in which porous hydrophilized PTFE membrane is used as a skeleton and a hydrogel (e.g., HM alginate) is used as filler. The alginate fills all the pore volume. As the pores volume of this membrane is small (typical maximum pore diameter is 0.4 μm) but their surface area high, the gel contained within the pores is easily stabilized by hydrophilic interactions.

In some embodiments, the immune protection membrane prevents immune cells, viruses and molecules form evading into the at least one hydrogel layer, without affecting the diffusion of oxygen and/or nutrients to the transplanted cells.

In some embodiments, the immune protection membrane prevents immune cells, viruses and molecules form evading into the at least one hydrogel layer, without affecting the diffusion of waste products/and or metabolites out of the device.

In some embodiments, the immune protection membrane prevents immune cells, viruses and molecules form evading into the at least one hydrogel layer, without affecting the viability and/or functionality of the transplanted cells.

In some embodiments, the immune protection membrane prevents immune cells, viruses and molecules form evading into the at least one hydrogel layer, without affecting the diffusion of insulin or glucose.

In some embodiments, the immune protection membrane may be dried by lyophilization and stored. The storage temperature may be 4 to 25 degrees Celsius. In some embodiments, the immune protection membrane may be re-hydrated, prior to incorporation into the device according to some embodiments of the present invention.

In some embodiments, the device comprises an oxygen supply container, having a chamber, defined by a top surface, a bottom surface, and sides, disposed within the chamber of the housing, wherein the top surface and the bottom surface of the oxygen supply container comprise at least one gas-permeable membrane. In some embodiments, the at least one gas-permeable membrane comprises silicone rubber-teflon. In some embodiments, the at least one gas-permeable membrane is the membrane disclosed in U.S. Pat. No. 8,821,431 B2.

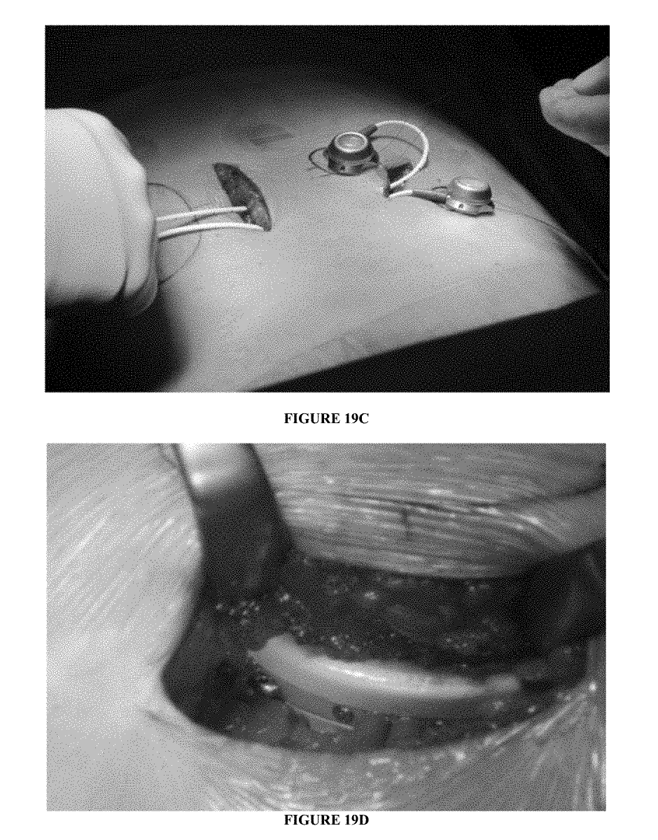

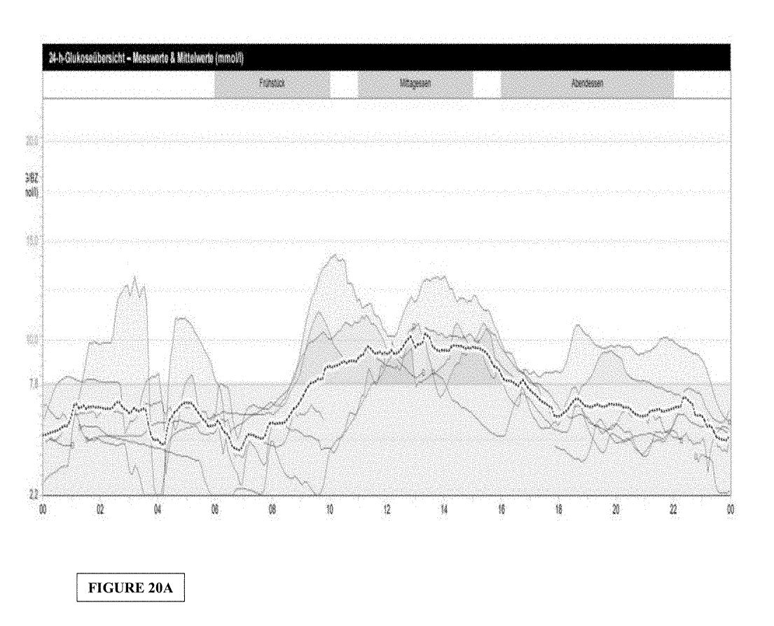

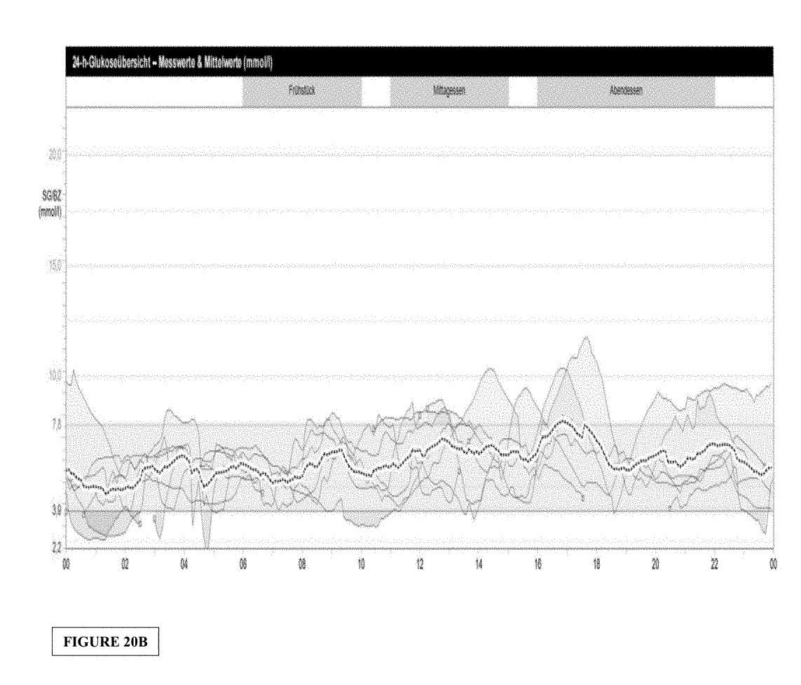

In some embodiments, the device of the present invention further comprises at least one port, configured to deliver oxygen to the chamber of the oxygen supply container, wherein the at least one port is fluidly connected to the chamber of the oxygen supply container; and at least one access port, configured to receive an exogenous supply of gas, fluidly connected to the at least one port. An example is shown in FIGS. 13 and 19C.

In some embodiments, the device is implanted into the body of the subject at a location selected from the group consisting of: a subcutaneous location, an intramuscular location, an intraperitoneal location, a pre-peritoneal location, and an omental location.

In some embodiments, the at least one access port is implanted remotely from the apparatus. In one embodiment, the at least one access port is implanted into the body of the subject at a location selected from the group consisting of: a subcutaneous location, an intramuscular location, an intraperitoneal location, a pre-peritoneal location, and an omental location.

In some embodiments, the device is implanted into the body of the subject according to the methods disclosed in Barkai et al., PLoSONE. In some embodiments, the device is implanted into the body of the subject according to the methods disclosed in Ludwig et al., PNAS.

In some embodiments, oxygen is delivered to the chamber of the oxygen supply container in an amount sufficient to maintain the viability and/or the functionality of the transplanted cells.

In some embodiments, the at least one access port is implanted subcutaneously and allowing for exogenous delivery of oxygen to the oxygen supply container using a transcutaneous needle. In some embodiments, the oxygen is delivered according to the methods described in U.S. Pat. No. 8,784,389 B2.

In some embodiments, the oxygen is delivered to the chamber of the oxygen supply container at an initial partial pressure of between 400-650 mmHg. In some embodiments, the oxygen is delivered to the chamber of the oxygen supply container at an initial partial pressure of between 450-650 mmHg. In some embodiments, the oxygen is delivered to the chamber of the oxygen supply container at an initial partial pressure of between 500-650 mmHg. In some embodiments, the oxygen is delivered to the chamber of the oxygen supply container at an initial partial pressure of between 550-650 mmHg. In some embodiments, the oxygen is delivered to the chamber of the oxygen supply container at an initial partial pressure of between 600-650 mmHg. In some embodiments, the oxygen is delivered to the chamber of the oxygen supply container at an initial partial pressure of between 400-600 mmHg. In some embodiments, the oxygen is delivered to the chamber of the oxygen supply container at an initial partial pressure of between 400-550 mmHg. In some embodiments, the oxygen is delivered to the chamber of the oxygen supply container at an initial partial pressure of between 400-500 mmHg. In some embodiments, the oxygen is delivered to the chamber of the oxygen supply container at an initial partial pressure of between 400-450 mmHg. In some embodiments, the oxygen is delivered to the chamber of the oxygen supply container at an initial partial pressure of between 450-600 mmHg. In some embodiments, the oxygen is delivered to the chamber of the oxygen supply container at an initial partial pressure of between 500-550 mmHg.

In some embodiments of the present invention, the device comprises a gas mixture comprising oxygen at a concentration of between 40% and 95% (e.g., but not limited to, 40%, 45%, 50%, 55%, etc.) and balance of nitrogen. In some embodiments, the oxygen mixture comprises 5% carbon dioxide. In some embodiments, the pressure of the gas mixture in the oxygen supply container is between 1.0 atm (ambient pressure) and 10 atm. In some embodiments, the pressure of the gas mixture in the oxygen supply container is between 5.0 atm (ambient pressure) and 10 atm. In some embodiments, the pressure of the gas mixture in the oxygen supply container is between 1.0 atmosphere (atm) (ambient pressure) and 5 atm. In some embodiments, the source of oxygen comprises approximately 5% carbon dioxide in order to maintain a balance of acidity of pH 7.4 between the inside of the housing and the body.

In some embodiments, oxygen is delivered once every 24 hours. In some embodiments, oxygen is delivered at least once every 24 hours. In some embodiments, oxygen is delivered at least once every 7 days or every 14 days.

In some embodiments, a gas mixture containing between 50 mm Hg and 500 mm Hg oxygen, 53 mm Hg CO₂ and balance of nitrogen is delivered into the oxygen supply container through the at least one access port. In some embodiments, the gas mixture delivered into the oxygen supply container through the at least one access port contains about 5% CO₂ (40 mm Hg). This level of CO₂ in the gas phase is in equilibrium with the bicarbonate in the tissue, resulting in acidity level of pH7.4. Therefore, no gradient will accrue between the oxygen supply container and the surrounding recipient tissue, thus not disturbing normal tissue acidity level.

Without intending to be limited by any particular theory, it is hypothesized that to supply oxygen from the oxygen supply container to the transplanted cells, oxygen diffuses through the at least one gas-permeable membrane, dissolving in the at least one hydrogel layer surrounding the transplanted cells, or dissolved in the matrix surrounding the cells (e.g. extracellular matrix, ECM) and diffuses to the transplanted cells. As oxygen diffuses into the hydrogel, or ECM its concentration decreases. Therefore, the oxygen concentration in the oxygen must by high enough to compensate for consumption by cells and loss to the surrounding tissue.

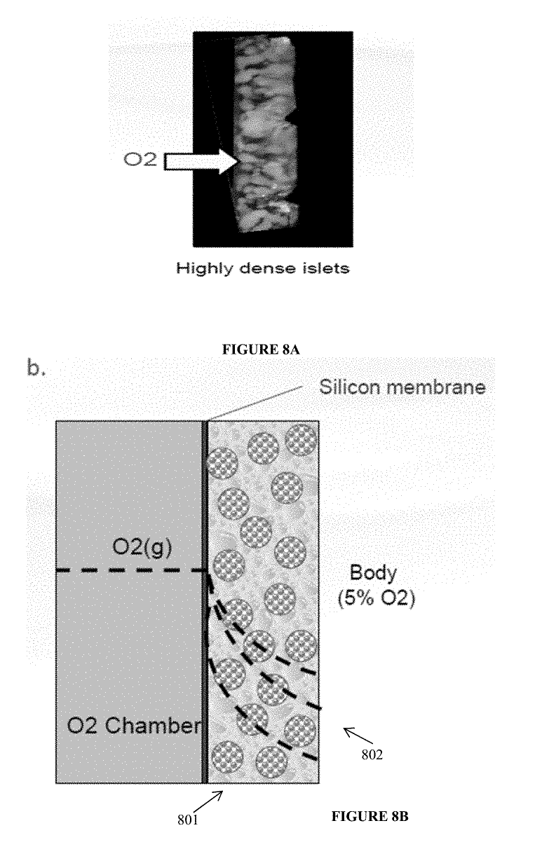

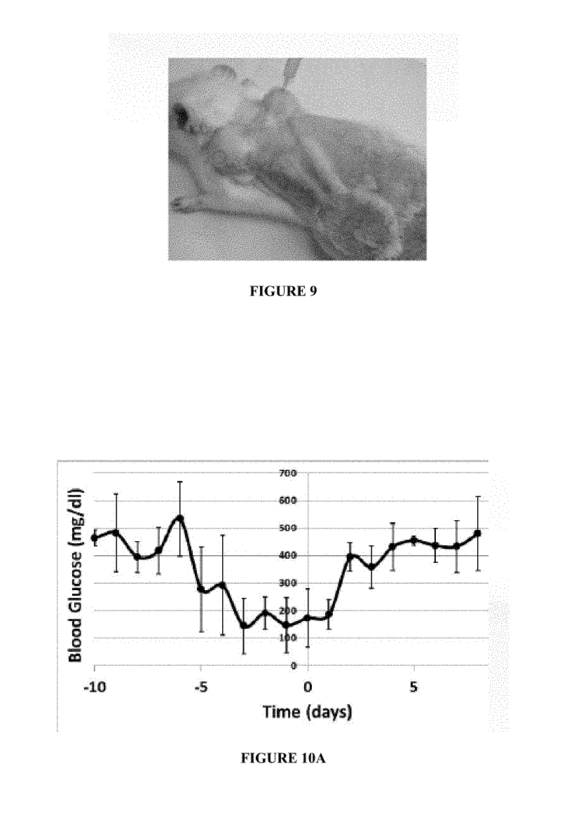

Referring to FIG. 8 B shows the theoretical oxygen gradient through the at least one hydrogel layer (FIG. 8A), illustrating low O₂ demand (e.g. lower cell density, upper broken line), or higher O₂ demand (e.g. higher cell density, lower broken line). In order to achieve maximum cell functionality, the lower O₂ concentration must be maintained around 50-60 mmHg. Therefore, the lowest O₂ at 802 should be 50-60 mmHg. In order to achieve the minimum of 50-60 mmHg, the inlet (801) must have a higher O₂ concentration. (801) is a surface adjacent to the oxygen supply container, while (802) is a surface adjacent to the subject's body.

In some embodiments, the device of the present invention is configured to provide the transplanted cells with at least 5% oxygen at the outer surface of the at least one hydrogel layer (FIG. 8B, 802).

In some embodiments, the device may be implanted permanently. Alternatively, the device may be removed after a period of time. The period of time may be greater than one year, one year, or less than one year. The period of time may be 1, 2, 3, 4, 5, 6, 7, 8, 9, 10, or 11 months.

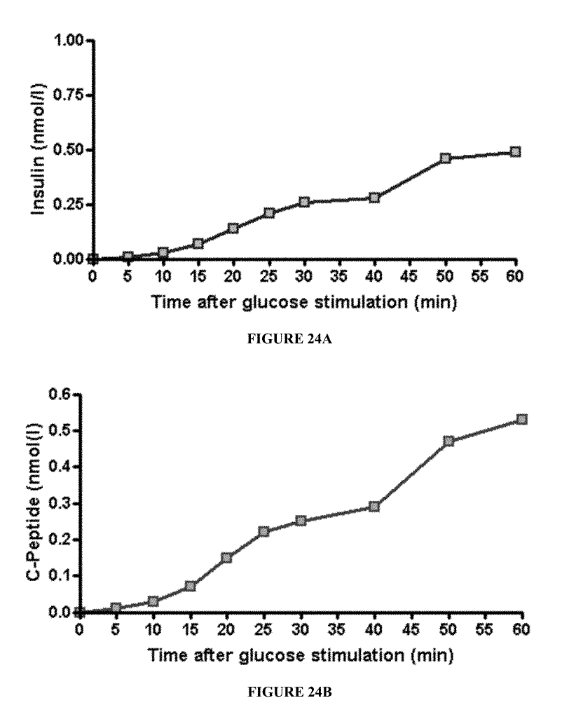

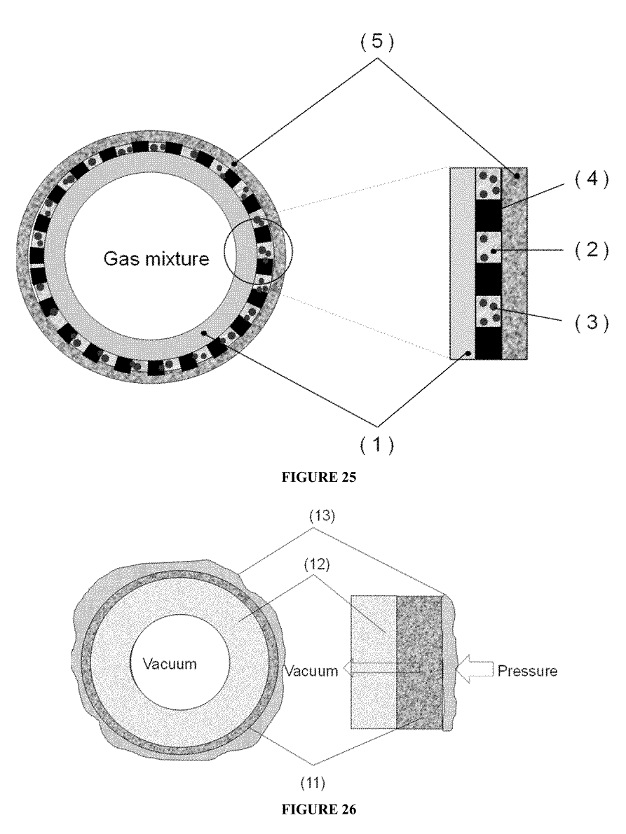

In some embodiments, the device of the present invention is the device shown in FIGS. 25-30. Referring to FIG. 25, a schematic illustration of a cross-section through an embodiment of the device of the present invention is shown. The device includes an internal gas mixture supply container which is a central cavity formed by a gas permeable membrane (1) which separates the internal cavity from a tissue or cell compartment (2). In an embodiment, the thickness of the gas permeable membrane is 10-400 μm. The internal supply container is flexible and sufficiently designed to hold a gas mixture. In an embodiment, the gas permeable membrane is a silicon rubber membrane. In an embodiment, the gas mixture contains oxygen, 5% CO₂, and balance of nitrogen. The gases are diffused via the gas permeable membrane into the cell compartment, which comprises a hydrogel (2) surrounding the transplanted cells (3). A rigid mesh (4) is designed to act as a mechanical skeleton to strengthen the bioartificial implant device and to maintain constant thickness for the gel (2), which holds the transplanted cells (3). The device further includes a composite membrane (5), composed of a hydrophilic porous membrane as the skeleton filled/impregnated with cross-linked gel.

Referring to FIG. 26, a schematic illustration of a first step of a first embodiment for manufacturing an impregnated gel membrane (composite membrane (5)) of a device according to some embodiments of the present invention is shown. A hydrophilized porous membrane (11), such as hydrophilized 0.4 μm porous polytetrafluoroethylene (PTFE) membrane (Biopore, Millipore; Schwalbach, Germany) is thread on a rigid porous material (12) such as sinter glass or porous stainless steel tubes) and located in a gel solution (13), such as high mannuronic (HM) alginate. A vacuum is activated inside the porous rigid tube (12), or pressure is activated on top of the gel (13) and the gel (13) penetrates into the void volume within the porous hydrophilized membrane (11). The excess gel is gently removed. The porous rigid tube (12) with the porous hydrophilized membrane (11) comprising the gel is immersed in a solution containing a cross linking agent such as barium, calcium, or strontium), dried by lyophilization and sterilized by low temperature ethylene oxide (ETO). In some embodiments, the solvent is histidine-tryptophanketoglutarate (HTK) and the solution has a final concentration of about 6% (w/v) of HM alginate.

FIG. 27 shows a schematic illustration of a cross-section through the dried composite membrane (5) produced according to the steps outlined in FIG. 26, composed of hydrophilized porous membrane as a skeleton and a hydrogel as a filler. In some embodiments, the composite membrane is manufactured using some or all of the steps described in the Materials and Methods portion of Neufeld et al. “The Efficacy of an Immunoisolating Membrane System for Islet Xenotransplantation in Minipigs”, PLOS ONE, August 2013, Vol. 8, Issue 8.

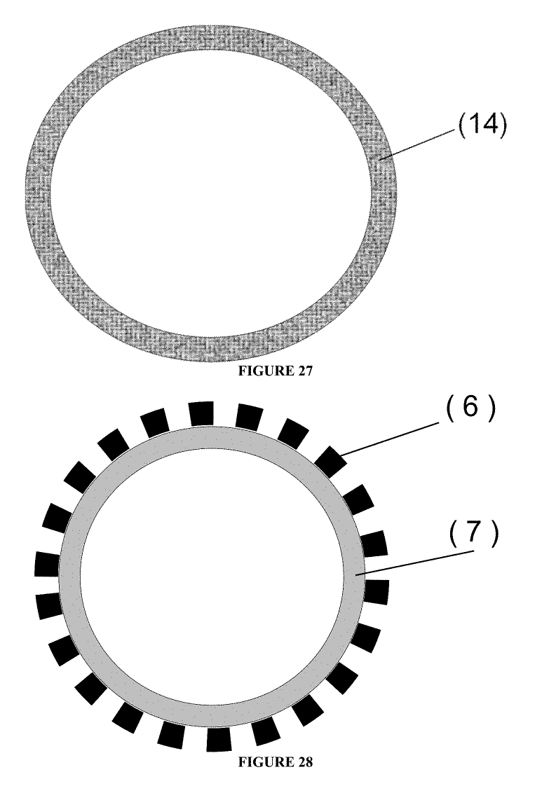

Referring to FIG. 28, a schematic illustration of a second step of a first embodiment for manufacturing a device according to some embodiments of the present invention is shown, in which a rigid mesh (6) is thread on a gas permeable tube (7). The thickness of the rigid mesh (6) is varied between about 10 μm and about 2,000 μm. In some embodiments, the rigid mesh (6) has a thickness of between about 100 μm and about 1,000 μm. In some embodiments, the rigid mesh (6) is made from a rigid material suitable for long-term implantation.

Examples of rigid materials suitable for use as a rigid mesh of the present disclosure include, but are not limited to, stainless-steel, PEEK (polyether ether ketone), and Nitinol. The void volume of the rigid mesh (6) allows maximum loading of the gel with the tissue and is varied between 10:1 (void volume to mesh volume) to 100:1 (void volume to mesh volume).

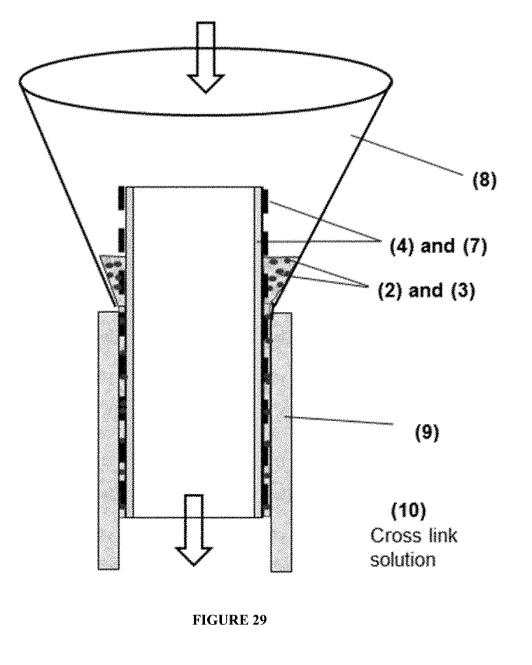

Referring to FIG. 29, a schematic illustration of a third step of a first embodiment for manufacturing a device according to some embodiments of the present invention is shown, in which a constant thickness amount of cells are immobilized on the gas permeable tube (7)/rigid mesh (4) construct. The gas permeable tube (7) covered with the rigid mesh (4) is inserted into an extrusion tool (8), which is composed of a conical funnel connected to porous tube (9), (e.g. sinter glass). Cells (3) mixed with gel (2) is poured around the gas permeable tube (7) and the rigid mesh (4) and the tube (7) and mesh (4) are pulled down into a rigid porous tube (9), (e.g. sinter glass). In some embodiments, the gel is selected from the group consisting of agarose, alginate and cellulose. In some embodiments, the gel is high guluronic acid (HG) alginate which has been dissolved in sterile water to a concentration of 0.5%, filter-sterilized through a membrane and freeze dried by lyophilization. The freeze dried HG alginate was dehydrated with histidine-tryptophanketoglutarate (HTK) to a concentration of between about 0.5% and about 5%. In some embodiments, the thickness of the gel comprising the transplanted cells is dictated by the rigid mesh (4), with a thickness between about 10 μm and about 1,000 μm.

In some embodiments, the cells (3) and gel (2) are mixed and applied between the extrusion tool (8) and the gaps in the rigid mesh (4). The gas permeable tube (7) and the rigid mesh (4) are pulled down, resulting in a uniform thickness of the cells (3) and gel (7). A solution containing a cross linker agent (10) such as barium, calcium, or strontium, is introduced around the pours rigid tube (9), resulting in solidification of the gel. The cells (3) and gel (2) fill up the spaces (void volume) between the rigid mesh (4).

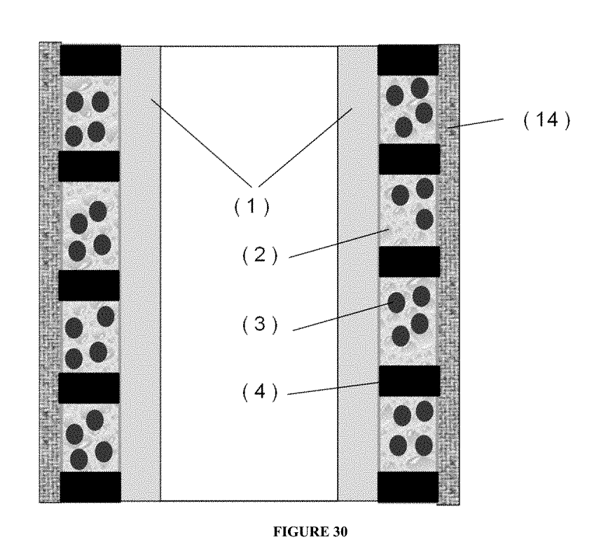

Referring to FIG. 30, a schematic illustration of a fourth step of a first embodiment for manufacturing a device according to some embodiments of the present invention is shown, in which the composite membrane (14) is thread on the device made of gas permeable membrane (1), tissue or cells (3) and rigid mesh (4). During the process of applying the dry composite membrane (14) on the device, the composite membrane becomes wet.

In some embodiments, the device comprises a thin layer of transplanted cells embedded in a cylindrical or ellipsoid hydrogel surrounding a flexible oxygen supply container, and separated from body liquids by a composite membrane allowing the transfer of small water soluble molecules such as glucose and insulin, and preventing the transfer of large water soluble molecules that implement immune response, such as immunoglobulins and complement components.

In some embodiments, the device is sufficiently designed so that oxygen gas passes from the interior of the flexible oxygen supply container, dissolves in the hydrogel, and diffuses into the transplanted cells. In an embodiment, the oxygen supply container includes a flexible gas permeable tube made of gas permeable materials. In some embodiments, the gas permeable material is silicon rubber. In some embodiments, the flexible gas permeable tube has a thickness of between about 1.0 μm and about 2,000 μm.

In some embodiments, the oxygen concentration in the contained gas is between 40 mmHg and 2,000 mmHg (the pressure of the gas might be over 1 ATM). In some embodiments, a CO₂ concentration in the chamber of the flexible oxygen supply container is 40 mmHg. In some embodiments, the composite membrane is made of porous hydrophilic membrane, such as PTFE hydrophilic membrane, as a skeleton having its void volume comprising alginate, such as, for example, HM alginate, as filler cross-linked with divalent ion, such as barium, strontium and calcium. In an embodiment, the composite membrane is dried before integrating on the device. In an embodiment, the composite membrane is sterilized by low temperature, for example between 32° C. and 36° C., ethylene oxide to prevent damage to the impregnate HM alginate.

The at Least One Hydrogel Layer

In some embodiments, the at least one hydrogel layer has a uniform thickness of between 100-700 micrometers. In some embodiments, the at least hydrogel layer has a uniform thickness of between 100-600 micrometers. In some embodiments, the at least one hydrogel layer has a uniform thickness of between 300-500 micrometers. In some embodiments, the at least one guluronic acid alginate layer has a uniform thickness of between 300-400 micrometers. In some embodiments, the at least one hydrogel layer has a uniform thickness of between 400-800 micrometers. In some embodiments the at least one hydrogel layer has a uniform thickness of between 500-800 micrometers. In some embodiments the at least one hydrogel layer has a uniform thickness of between 600-800 micrometers. In some embodiments, the at least one hydrogel layer has a uniform thickness of between 700-800 micrometers. In some embodiments, the at least one hydrogel layer has a uniform thickness of between 400-700 micrometers. In some embodiments, the at least one hydrogel layer has a uniform thickness of between 500-600 micrometers.

In some embodiments, the at least one hydrogel layer comprises guluronic acid alginate.

In some embodiments, the at least one hydrogel layer is generated according to the methods disclosed in U.S. Patent Application Publication No. 20110165219 A1. In some embodiments, the at least one hydrogel layer is generated according to the methods disclosed in Neufeld et al., PLoSONE. In some embodiments, the at least one hydrogel layer is generated according to the methods disclosed in Ludwig et al., PNAS.

In some embodiments, the at least one hydrogel layer is supported by a mesh.

The Transplanted Tissue

In some embodiments, the device of the present invention comprises transplanted cells contained within at least one hydrogel layer.

In some embodiments, the transplanted cells are contained within the at least one hydrogel layer according to the methods described in U.S. Patent Application Publication No. 20110165219 A1. In some embodiments, the transplanted cells are contained within the at least one hydrogel layer according to the methods described in Neufeld et al., PLoSONE. In some embodiments, the transplanted cells are contained within the at least one hydrogel layer according to the methods described in Ludwig et al., PNAS.

In some embodiments, the transplanted cells are selected from the group consisting of islets of Langerhans, stem cells, adrenal cells, insulin secreting cells, beta cells, alpha cells, stem cell-derived insulin producing cells, stem cell-derived beta cells, stem cell-derived alpha cells and genetically modified cells.

In some embodiments, the transplanted cells are allogeneic. In some embodiments, the transplanted cells are xenogeneic. In some embodiments, the transplanted cells are isogeneic. In some embodiments, the transplanted cells are autologous.

In some embodiments, the transplanted cells comprise isolated pancreatic islets. Isolation of the pancreatic islets may be carried out via enzymatic digestion of donor Pancreata, for example, according to the methods described in Matsumoto et al., Proc (Bayl. Univ. Med. Cent.). 2007 October; 20(4): 357-362.

In some embodiments, the transplanted cells contained within the at least one hydrogel layer has a density between 1,000 IEQ/cm² and 15,000 IEQ/cm². In some embodiments, the transplanted cells contained within the at least one hydrogel layer has a density between 1,000 IEQ/cm² and 14,000 IEQ/cm². In some embodiments, the transplanted cells contained within the at least one hydrogel layer has a density between 1,000 IEQ/cm² and 13,000 IEQ/cm². In some embodiments, the transplanted cells contained within the at least one hydrogel layer has a density between 1,000 IEQ/cm² and 12,000 IEQ/cm². In some embodiments, the transplanted cells contained within the at least one hydrogel layer has a density between 1,000 IEQ/cm² and 11,000 IEQ/cm². In some embodiments, the transplanted cells contained within the at least one hydrogel layer has a density between 1,000 IEQ/cm² and 9,000 IEQ/cm². In some embodiments, the transplanted cells contained within the at least one hydrogel layer has a density between 1,000 IEQ/cm² and 8,000 IEQ/cm². In some embodiments, the transplanted cells contained within the at least one hydrogel layer has a density between 1,000 IEQ/cm² and 7,000 IEQ/cm². In some embodiments, the transplanted cells contained within the at least one hydrogel layer has a density between 1,000 IEQ/cm² and 6,000 IEQ/cm². In some embodiments, the transplanted cells contained within the at least one hydrogel layer has a density between 1,000 IEQ/cm² and 5,000 IEQ/cm².

In some embodiments, the transplanted cells contained within the at least one hydrogel layer has a density between 1,000 IEQ/cm² and 4,800 IEQ/cm². In some embodiments, the transplanted cells contained within the at least one hydrogel layer has a density between 2,400 IEQ/cm² and 4,800 IEQ/cm². In some embodiments, the transplanted cells contained within the at least one hydrogel layer has a density between 3,600 IEQ/cm² and 4,800 IEQ/cm². In some embodiments, the transplanted cells contained within the at least one hydrogel layer has a density between 1,000 IEQ/cm² and 3,600 IEQ/cm². In some embodiments, the transplanted cells contained within the at least one hydrogel layer has a density between 1,000 IEQ/cm² and 2,400 IEQ/cm². In some embodiments, the transplanted cells contained within the at least one hydrogel layer has a density between 2,400 IEQ/cm² and 3,600 IEQ/cm².

In some embodiments, the transplanted cells comprise stem cell-derived insulin producing cells. In some embodiments, the stem cell-derived insulin producing cells are the cells disclosed in U.S. Pat. No. 8,338,170. In some embodiments, the stem cell-derived insulin producing cells are the cells disclosed in U.S. Pat. No. 8,859,286. In some embodiments, the stem cell-derived insulin producing cells are the cells disclosed in U.S. Pat. No. 9,109,245.

In some embodiments, the transplanted cells contained within the at least one hydrogel layer has a density between 1,000,000 cells/cm² and 100,000,000 cells/cm². In some embodiments, the transplanted cells contained within the at least one hydrogel layer has a density between 2,000,000 cells/cm² and 100,000,000 cells/cm². In some embodiments, the transplanted cells contained within the at least one hydrogel layer has a density between 3,000,000 cells/cm² and 100,000,000 cells/cm². In some embodiments, the transplanted cells contained within the at least one hydrogel layer has a density between 4,000,000 cells/cm² and 100,000,000 cells/cm². In some embodiments, the transplanted cells contained within the at least one hydrogel layer has a density between 5,000,000 cells/cm² and 100,000,000 cells/cm². In some embodiments, the transplanted cells contained within the at least one hydrogel layer has a density between 6,000,000 cells/cm² and 100,000,000 cells/cm². In some embodiments, the transplanted cells contained within the at least one hydrogel layer has a density between 7,000,000 cells/cm² and 100,000,000 cells/cm². In some embodiments, the transplanted cells contained within the at least one hydrogel layer has a density between 8,000,000 cells/cm² and 100,000,000 cells/cm². In some embodiments, the transplanted cells contained within the at least one hydrogel layer has a density between 9,000,000 cells/cm² and 100,000,000 cells/cm². In some embodiments, the transplanted cells contained within the at least one hydrogel layer has a density between 10,000,000 cells/cm² and 100,000,000 cells/cm². In some embodiments, the transplanted cells contained within the at least one hydrogel layer has a density between 10,800,000 cells/cm² and 100,000,000 cells/cm².

In some embodiments, the transplanted cells contained within the at least one hydrogel layer has a density between 1,000,000 cells/cm² and 90,000,000 cells/cm². In some embodiments, the transplanted cells contained within the at least one hydrogel layer has a density between 1,000,000 cells/cm² and 80,000,000 cells/cm². In some embodiments, the transplanted cells contained within the at least one hydrogel layer has a density between 1,000,000 cells/cm² and 70,000,000 cells/cm². In some embodiments, the transplanted cells contained within the at least one hydrogel layer has a density between 1,000,000 cells/cm² and 60,000,000 cells/cm². In some embodiments, the transplanted cells contained within the at least one hydrogel layer has a density between 1,000,000 cells/cm² and 50,000,000 cells/cm². In some embodiments, the transplanted cells contained within the at least one hydrogel layer has a density between 1,000,000 cells/cm² and 40,000,000 cells/cm². In some embodiments, the transplanted cells contained within the at least one hydrogel layer has a density between 1,000,000 cells/cm² and 30,000,000 cells/cm². In some embodiments, the transplanted cells contained within the at least one hydrogel layer has a density between 1,000,000 cells/cm² and 20,000,000 cells/cm². In some embodiments, the transplanted cells contained within the at least one hydrogel layer has a density between 1,000,000 cells/cm² and 19,200,000 cells/cm².

In some embodiments, the transplanted cells contained within the at least one hydrogel layer has a density between 10,800,000 cells/cm² and 19,200,000 cells/cm². In some embodiments, the transplanted cells contained within the at least one hydrogel layer has a density of a value between 12,000,000 cells/cm² and 19,200,000 cells/cm². In some embodiments, the transplanted cells contained within the at least one hydrogel layer has a density between 14,000,000 cells/cm² and 19,200,000 cells/cm². In some embodiments, the transplanted cells contained within the at least one hydrogel layer has a density of a value between 16,000,000 cells/cm² and 19,200,000 cells/cm². In some embodiments, the transplanted cells contained within the at least one hydrogel layer has a density between 18,000,000 cells/cm² and 19,200,000 cells/cm². In some embodiments, the transplanted cells contained within the at least one hydrogel layer has a density between 10,800,000 cells/cm² and 18,000,000 cells/cm². In some embodiments, the transplanted cells contained within the at least one hydrogel layer has a density between 10,800,000 cells/cm² and 16,000,000 cells/cm². In some embodiments, the transplanted cells contained within the at least one hydrogel layer has a density between 10,800,000 cells/cm² and 14,000,000 cells/cm². In some embodiments, the transplanted cells contained within the at least one hydrogel layer has a density between 10,800,000 cells/cm² and 12,000,000 cells/cm². In some embodiments, the transplanted cells contained within the at least one hydrogel layer has a density between 12,000,000 cells/cm² and 18,000,000 cells/cm². In some embodiments, the transplanted cells contained within the at least one hydrogel layer has a density between 14,000,000 cells/cm² and 16,000,000 cells/cm².

In some embodiments, the transplanted cells can survive in the implantable medical device according to some embodiments of the present invention for at least a month, two months, three months, four months, five months, six months, seven months, eight months, nine months, ten months, eleven months, twelve months or a year or more.

In some embodiments, the transplanted cells retain at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more of their initial viability.

In some embodiments, the transplanted cells retain at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more of their initial density.

In some embodiments, the transplanted cells retain at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more of their initial functionality.

In some embodiments, the transplanted cells may further differentiate, or mature following introduction into the implantable medical device according to some embodiments of the present invention. Examples include, but are not limited to, implantation of progenitor cells, which further develop or mature to functional cells. The further differentiation may occur prior to implantation of the implantable medical device according to some embodiments of the present invention into a recipient. Alternatively, the further differentiation may occur after implantation of the implantable medical device according to some embodiments of the present invention into a recipient.

In some embodiments, the density, or, alternatively, the amount of the transplanted cells may increase (such as, for example, via cell division). The density, or amount may increase prior to implantation of the implantable medical device according to some embodiments of the present invention into a recipient. Alternatively, the density, or amount may increase after implantation of the implantable medical device according to some embodiments of the present invention into a recipient. In some embodiments, the recipient is a subject in need of treatment.

In some embodiments, the transplanted cells may self-renew (i.e., replace transplanted cells lost due to death) via cell division.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

Reference is now made to the following examples, which together with the above descriptions illustrate some embodiments of the invention in a non-limiting fashion.

### EXAMPLES

### Example 1: Treatment of Diabetic Rats with a Device According to Some Embodiments of the Present Invention

### Materials and Methods

Animals, Induction of Diabetes, and Pre-Treatment:

Lewis rats (260-280 g) were purchased from Harlan (Rehovot, Israel), and diabetes was induced by a single intravenous infusion of 85 mg/Kg body weight of Streptozotocin (STZ; Sigma, Israel). Animals had free access to food at all times and were considered diabetic when non-fasting blood glucose exceeded 450 mg/dl for 4 consecutive days or more.

To prepare the diabetic animals for device implantation in a non-stressing, normal blood glucose environment, 1.5 capsules of a sustained release insulin implant (Linplant, LinShin, Toronto, Canada) were inserted under the skin of the diabetic animals, which were considered ready for implantation of the device when their non-fasted blood glucose was under 250 mg/dL for 3 consecutive days or more. The sustained release insulin capsules were removed 48 hr after implantation, leaving the encapsulation device as the only source for insulin, following implantation according to the methods described below. The efficacy of glycemic control was followed for 60 days after implantation, by assessing the functionality of the islets in the device through twice daily measurements of non-fasting blood glucose concentration. Animals were sedated, blood samples were collected from the tail, and glucose levels were measured by commercial glucometer (Accu-Chek sensor, Roche Diagnostics GmbH). Intravenous glucose tolerance tests (IVGTT) were performed 6 weeks post-transplantation as follows: animals were fasted overnight. On the following morning, 1 ml of 0.7M glucose solution was infused within 10-15 sec (dose of 500 mg/kg BW), and blood glucose samples were collected for measurement before infusion and at 10, 30, 60, 120 and 180 min following glucose infusion.

Islet Isolation and Culture:

Pancreata were obtained from 9 to 10-week old male Lewis rats weighing 260-280 g and underwent collagenase digestion of the donor pancreata. Briefly, each pancreas was infused with 10 ml enzymatic digestive blend containing 15 PZ units collagenase NB8 (Serva, Heidelberg, Germany) and 1 mg/ml bovine DNAse (Sigma, cat. no. 159001) in Hank's balanced salt solution (HBSS; Biological Industries, Bet HaEmek, Israel) for 14 min. Islets were purified on discontinuous Histopaque gradient [1.119/1.100/1.077/RPMI (Sigma)] run for 20 min at 1,750 g/max in the cold (6° C.). Islets were then washed twice and cultured in complete CR medium [Connaught Medical Research Laboratories (CMRL): Roswell Park Memorial Institute (RPMI) medium (1:1) supplemented with 10% fetal bovine serum (Bet-HaEmek, Israel)] for 1 week prior to being integrated in implantable devices.

For determining a number of cells in a rat islet equivalent (IEQ), 21 different lots containing 50-60 standard islets each were selected. Islets were defined as “standard” when diameters of both x- and y-axes were estimated to be 150 For enumeration of cells, the method of DTZ staining, as described herein, was used and a value of 1,556±145 cells/IEQ was obtained. Immediately after isolation, rat islets were subjected to the same enumeration protocol and found to contain 1,430±185 cells/islet (n=10). At the time of device assembly (6-8 days after isolation), we enumerated 1,270±280 cells/IEQ (n=107). During the cultivation period, cell count in an average islet declined by <20%, and therefore, at time of implantation, an islet particle was estimated to correspond to 0.8 IEQ. Islet particles were either isogeneic (i.e., derived from Lewis rats and implanted into diabetic Lewis rats) or allogeneic (i.e., derived from Sprague-Dawley rats and implanted into diabetic Lewis rats).

Islet Enumeration by Conventional Counting with DTZ Staining:

Two representative aliquots of 100 μl each from the final islet preparation were incubated with DTZ working solution as described for volume fraction determination by DTZ staining. Using a light microscope with a Bausch and Lomb micrometer disc (31-16-08) eyepiece reticle containing a grid of squares 50 μm on a side, the number of squares and the area occupied by each stained islet was determined, and the diameter of a circle having about the same surface area was estimated for each islet. Size distribution of the islets was quantified by two independent observers in 50 μm increments (ranges: 50-100, 100-150, 150-200, 200-250, 250-300, 300-350, and >350 μm). A formula was used to convert the number of islets in each 50 μm increment to a total islet volume by assuming that the islets are spherical. The number of IEs was calculated as the total islet volume divided by the volume of an IEQ (1.77×10⁶ μm³).

The Subcutaneously Implantable Device: The subcutaneously-implantable device had an external disc-shaped housing made of clinical grade polyether ether ketone (PEEK Optima LT1R40; Invibio, Lancashire, UK) with a diameter of 31.3 mm and thickness of 7 mm.

Referring to FIGS. 1A, 1B, and 1C, the device consisted of three major components: 1. The islet chamber contained about 2,400 islet equivalents (IEQ) embedded in 500 to 600-μm thick ultrapure high guluronic acid alginate layer, reinforced with 100-μm thick stainless steel grids having about 80% fractional open area (top grid, FIG. 1A, insert, Suron, Ma'agan Michael, Israel), glued to the PEEK housing with medical epoxy adhesive (Epotek 301-2 Billerica, Mass., USA). Mechanical support was provided by the bottom grid, identical to the top grid, which was placed under the gas permeable membrane and reinforced by PEEK mechanical supports (see FIG. 1 A). To vary islet density, 2,400 islets were immobilized in a hydrogel layer with a diameter of 18, 11.3, 9.8, or 8.0 mm, resulting in densities of 1,000, 2,400, 3,600, or 4,800 IEQ/cm² en face surface area for oxygen transport, respectively (see FIG. 1 B). 2. The oxygen supply container (3-ml volume) was separated from the islet module by a 25-μm gas-permeable silicone rubber-teflon membrane (Silon, BMS, Allentown, Pa.) and contained inlet and outlet oxygen supply container ports connected by two polyurethane tubes to subcutaneous access ports (Cat. No. PMINO-PU-C70, Instech Solomon, Pa.) implanted under the skin at a site remote from the device, as previously described (Barkai at. al., 2013). 3. A 25-μm, 0.4-μm pore diameter hydrophylized polytetrafluoroethylene (PTFE) membrane (Biopore, Millipore, Billerica, Mass.), separated the islet module from the body fluids and protected the islets from the cellular part of the immune system.

Device Assembly:

A dose of 2,400±200 IEQ was collected by 5-min sedimentation, The pellet was gently mixed with 2.2% (w/v) ultrapure high-guluronic acid (68%) alginate (Pronova UPMVG, Novamatrix; Sandvika, Norway), and the mixture was placed in the islet module compartment and spread through the openings of the top grid (see FIG. 1 A). The PTFE (Biopore) membrane was then fixed onto the device using a Viton O-Ring (hardness 75 Shore and outer diameter 27 mm), (McMaster Carr; Aurora, Ohio) and sealed to the plastic housing with medical silicone glue (MED 2000, Polytek Easton, Pa.). The alginate was cross-linked by applying a flat sintered glass (Pyrex, UK) saturated with strontium chloride dissolved in RPMI medium for a final concentration of 70 mM. The device and sintered glass were immersed in the RPMI-strontium medium for 16 min, resulting in a 500 to 600-μm thick coin-like hydrogel layer. The thickness variations originated with variation in glue thickness. The device was washed for an additional 5 min at 37° C. in complete CR medium (Beit HaEmek, Israel). Fully fabricated devices were washed in complete CR medium at 37° C. with agitation for 2 hours before implantation.

Device Implantation:

All animal experiments were performed according to guidelines established by the Israeli Institutional Animal Care and Use Committees. Rats were anesthetized by intraperitoneal injection of 90 mg/Kg Ketamine and 10 mg/Kg Xylazine followed by isoflurane inhalation. A 3-cm incision was made for the device on the dorsal skin, and muscles were separated from the hypodermis. A second incision was made in the skin between the shoulder blades, and two channels connecting this site with the device implantation site were created by traversing 3-mm wide stainless steel needles under the skin. The device was inserted under the dorsal skin incision with the islet module facing the fascia, and the oxygen supply container ports were connected to the remote subcutaneous access ports. The skin was sutured and fixed with a tissue adhesive (Histoacryl, Tufflingen, Germany).

Gas Mixture Replacement:

Every 24 hours the animal was sedated with isoflurane inhalation. A 27G needle was inserted into each of the two implanted access ports, and the oxygen supply container was purged with 20 ml (about 6.7 chamber volumes) of gas mixture containing the specified oxygen concentrations, 40 mmHg CO₂, and balance N₂. The final total pressure in the oxygen supply container was equal to ambient atmospheric pressure. To obtain the different oxygen mixtures, prefilled cylinders were used (Maxima, Israel).

Oxygen Consumption Rate:

Oxygen consumption rate (OCR) of post-implanted islets was determined following explantation of the device, release of the hydrogel layer from the device and manual counting of islets using doses of greater than or equal to 200 islets.

Islet OCR:

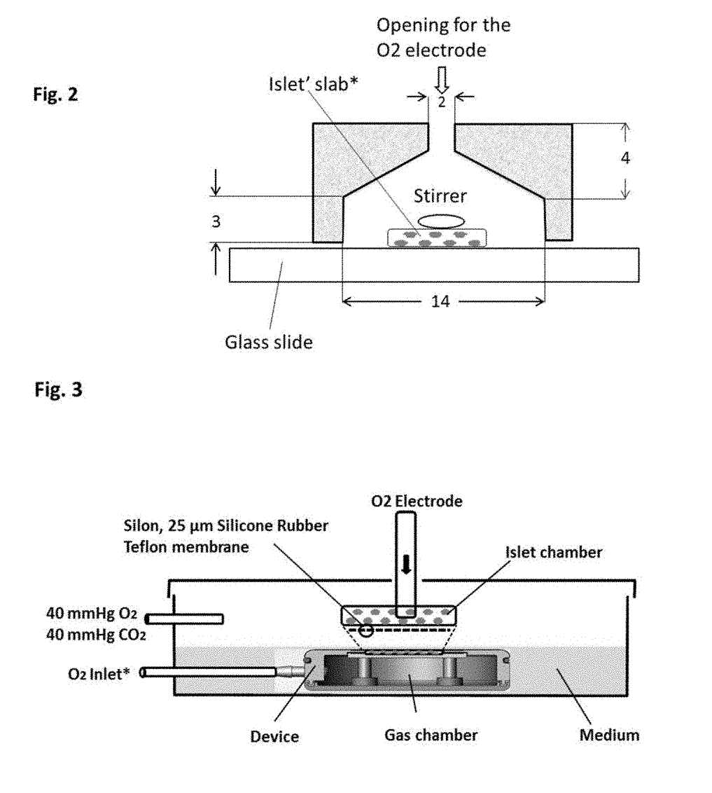

Preimplanted 250 IEQ immobilized in 30 μL of high guluronic acid alginate was shaped as a coin with a thickness of 500 μm. The hydrogel layer was placed on a glass slide with 5 mm diameter magnetic stirrer on top and covered with a conical OCR measurement chamber (FIG. 2). The conical chamber was filled with 1:1, CMRL:RPMI medium with 1% (v/v) fetal bovine serum to a final volume of 620 μl. The chamber was equipped with Clark-type oxygen electrode of 500-μm diameter connected to a picoamper controller (Cat No. PA2000, Unisense, Arhaus, Denmark). The O₂ measurement chamber was placed within a Perspex box with the air temperature maintained at 37±1° C. using a temperature control unit (Eurotherm 808; Eurotherm Worthing, UK). The stirring speed was increased until OCR did not change (about 70 rpm), assuring minimal effects associated with mass transfer boundary layers around the islets and the O₂ electrode. No damage to the hydrogel layer or the islets was observed as assessed by islet and hydrogel layer morphology and stable OCR readings. The electrode was calibrated using medium equilibrated with gas containing zero or ambient oxygen concentrations. The O₂ concentrations in both phases are reported here as oxygen partial pressure p, in units of mmHg, related to oxygen concentration c by the relation: c=αp, where α is the Bunsen solubility coefficient, 1.34×10⁻⁹ mole/(cm³ mmHg) for oxygen in medium at 37° C. Consequently, for example, at steady-state ambient O₂ partial pressure of 160 mmHg (21% O₂, 1 atm), dissolved O₂ concentration is 215 μM in the medium at 37° C. As a result of the O₂ consumption by the islets, the O₂ concentration in the medium within the conical measurement chamber decreased with time. The data for O₂ concentration with time was fitted by linear regression, and the slope was used to estimate OCR of the islets. The OCR was calculated from:

$$OCR = {V}_{ch} α ( \frac{Δ {pO}_{2}}{Δ t} ) \qquad ( Equation 1 )$$

where Vch is the chamber volume and a is the Bunsen solubility coefficient, taken to be 1.27 nmol/cm3·mm Hg at 37° C. Data above 60 mmHg in the region yielding the steepest slope of pO2 versus time was fitted to a straight line using linear regression. OCR per IEQ was obtained by dividing both sides of Equation (1) by the number of IEQ's (nC) in the chamber:

$$\frac{OCR}{IEQ} = \frac{α ( \frac{Δ {pO}_{2}}{Δ t} )}{\frac{{n}_{C}}{{V}_{ch}}} \qquad ( Equation 2 )$$

where the quantity nC/Vch is the cell concentration measured, for example, by nuclei counting. The quantity OCR/DNA can be calculated from Equation (2) if the denominator is replaced by DNA concentration in the chamber.

OCR Measurement after Removal of the Device:

Upon an elective removal of the device, the hydrogel layer containing the islets was carefully removed. Islets were counted under the microscope, the hydrogel layer was located in the OCR chamber (shown in FIG. 2) and the OCR was tested as described above.

Oxygen Gas Measurements:

To measure O₂ concentration in the oxygen supply container within the implanted devices, a 27G needle connected to 1.0 ml syringe was inserted into one of the implanted subcutaneous access ports, and a 250-μl sample was taken from the oxygen supply container 24 hr after the last O₂ replenishment and injected into the conical measurement chamber. The change in the electrode measurement was used to calculate the oxygen concentration in the sample from the oxygen supply container. The O₂ electrode was calibrated with gas containing zero O₂ concentration (pure N₂) and 160 mmHg (ambient air).

Oxygen Profile Across the Transplanted Islets:

About 2,400 IEQ were immobilized at various densities as described for subcutaneously-implantable device assembly, but without the PTFE (Biopore) membrane and without the metal grid on top. The device was placed in a covered 90 mm Petri-dish, overlain with RPMII medium so as to create a layer of minimal depth on top of the device, and the space above the hydrogel layer was purged with a gas stream having 40 mmHg O₂, 40 mmHg CO₂, and 680 mmHg N₂ (FIG. 3), which simulated the gas composition in the subcutis. The oxygen supply container was purged with oxygen concentrations varying between 152 and 304 mmHg. An O₂ electrode with a diameter of 500 μm, attached to a micromanipulator was inserted into the islet-containing hydrogel layer and advanced at 100 μm increments from the distal side of the islet-containing hydrogel layer downwards toward the gas permeable membrane. At each step, the O₂ electrode readings reached a steady-state level before moving to the next step. The entire measurement system was located in a 37° C. chamber. Data are expressed as mean±standard deviation. Statistical significance (p<0.05) was determined by the student's t-test.

Results:

Typically, as oxygen diffuses radially inward from the islet surface, oxygen is consumed by the cells in which it contacts. Accordingly, oxygen concentration decreases as it progresses toward the center of the islet. For a spherical islet equivalent (IEQ) of human origin, containing an average of 1,560 cells and having a diameter of 150 μm, the outer islet surface requires an oxygen partial pressure about 45-50 mmHg to maintain full functionality of all cells. As the density of the islets increased, the oxygen gradient across the at least one hydrogel layer increased. See, for example, FIG. 8 B.

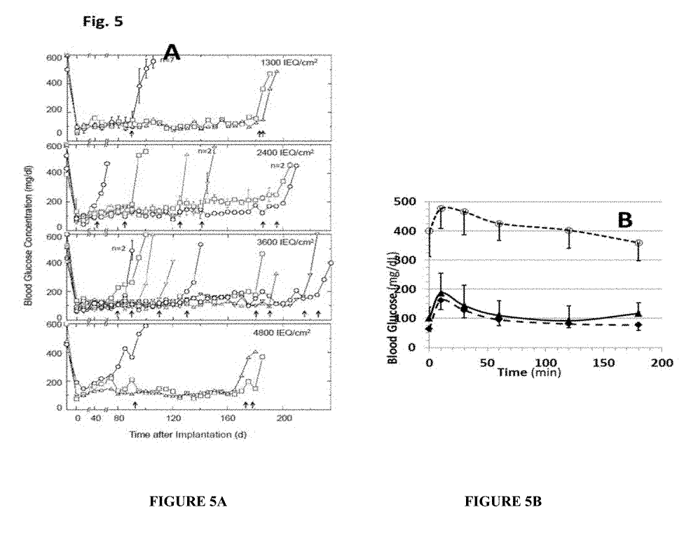

Islets were immobilized within the device in an alginate hydrogel layer having a thickness of between 500-600 μm. A gas mixture containing oxygen was supplied to the islets from an adjacent oxygen supply container by diffusion through a 25-μm gas-permeable membrane. The gas mixture in the chamber was replenished every 24 hours. In vitro experiments were used to determine the minimum initial O₂ concentration in the gas mixture loaded into the chamber that would support densities of islets as high as 4,800 IEQ/cm². The density of functional islets that could be supported increased with increasing O₂ concentration in the chamber. Devices containing various islet densities and sufficient oxygen supply container oxygen levels were implanted in streptozotocin-induced (“STZ-induced”) diabetic rats for up to 250 days. See FIG. 5 A. The rats achieved normoglycemia for the entire period and displayed near-normal responses to intravenous glucose tolerance tests. See FIG. 5 B. The data demonstrate the ability of the device to supply oxygen to implanted islets and to maintain islet viability and functionality at high islet immobilization densities (e.g., but not limited to, 4,800 IEQ/cm²). Accordingly, using the technology described herein, the required size of an implanted device suitable for human use can be substantially reduced.

pO2 in the Transplanted Islets:

The islets within the device were randomly scattered throughout the hydrogel layer. While some islets were located close to the O₂ source (i.e., the 25-μm silicone rubber-teflon gas-permeable membrane adjacent to the oxygen supply container, see, for example, FIG. 1 A insert), other islets were located far from the source (i.e., close to the device-tissue interface). To maintain a 150-μm islet fully functional, the minimal O₂ concentration on the surface of the islet should be above 50 mmHg.

To determine conditions that assure all islets were exposed to the required O₂ concentration, the in vitro test system shown in FIG. 3 used to measure the pO₂ profile across the transplanted islets by introducing an O₂ electrode to various depths within the transplanted islets.

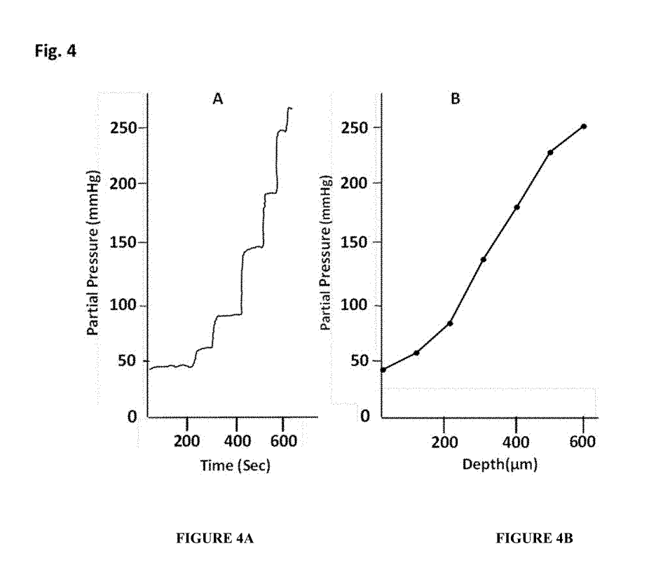

FIGS. 4A and 4B show a representative pO₂ profile within the transplanted islets following purging of the oxygen supply container with a gas mixture having a pO₂ of 304 mmHg while the medium above the transplanted islets was continuously purged with O₂ and CO₂, both at a concentration of 40 mmHg and the balance N₂. FIG. 4 A shows that after each incremental increase in pO₂, steady state was achieved in less than 30 seconds. FIG. 4 B shows that an increased pO₂ was required for islet survival when the distance from the oxygen source increased. The maximum value measured near the O₂-permeable membrane was about 260 mmHg; pO₂ decreased to a minimum of about 50 mmHg at the most distal part of the oxygen supply container (i.e., furthest from the oxygen source).

Minimum Oxygen Concentration Required in the Oxygen Supply Container at Various Islet Densities:

An increase in the density of the islets would result in a decrease in the oxygen-permeable surface area required (if all of the islets remained viable and functional), thereby leading ultimately to a smaller device for implantation. In this study, a fixed quantity of islets was packed in circular hydrogel layers of successively reduced diameter, area and volume (i.e., 18, 11.3, 9.8, or 8.0 mm, resulting in densities of 1,300, 2,400, 3,600, or 4,800 IEQ/cm²), which comprised the islet module of the device. The O₂ gradient across the hydrogel layer increased, resulting in lower O₂ concentration at the islet-containing hydrogel layer-tissue interface. To compensate for the increased islet density, the level of oxygen in the oxygen supply container was increased (see, e.g., Table 1).

The effect of islet density on the minimum level of pO₂ required in the oxygen supply container for keeping oxygen partial pressure at the most distal part of the hydrogel layer above 50 mmHg was measured with the in vitro system. Islets at different densities (2,400, 3,600, or 4,800 IEQ/cm²) were immobilized in hydrogel layers having a thickness of 500-600-μm. The oxygen supply container was purged with increased O₂ levels until the oxygen partial pressure at the outermost part of the hydrogel layer reached a value of about 50 mmHg (i.e., the surface furthest from the oxygen supply container). The corresponding pO₂ level in the oxygen supply container was designated the minimum pO₂, (Table 1). A pO₂ of 305 mmHg in the oxygen supply container was required to supply a high density (e.g., 4,800 IEQ/cm²) islet-containing hydrogel layer with adequate oxygen across the entire hydrogel layer thickness. The minimum oxygen concentration for cells (e.g., stem cells) is between 1.0-67 micromolar.

3)

Table 1: Minimum Oxygen Concentration in the Oxygen Supply Container Required for Functional Immobilized Islets.

Table 1 shows that about 2,400 IEQ with OCR between 3.4 and 3.8 pmol/IEQ/min were immobilized at various densities (i.e., 48 IEQ/cm³ (2,400 IEQ/cm²); 72 IEQ/cm³ (3,600 IEQ/cm²); and 96 IEQ/cm³ (4,800 IEQ/cm²)). The minimum pO₂ is the lowest oxygen concentration in the oxygen supply container required to achieve 50 mmHg at the interface between the islet-containing alginate hydrogel layer and the subcutaneous tissue. (N=3 experiments.)

| Islet density | Minimum pO₂ |
|---|---|
| (IEQ/cm³) | (mmHg) |
| 48 | 190 ± 31 |
| 72 | 229 ± 57 |
| 96 | 305 ± 34 |

Oxygen concentrations were sufficient to support the islets. The implantable device was designed for O₂ replenishment to be carried out every 24 hr. During this period, the O₂ concentration in the oxygen supply container would decrease as a result of oxygen consumption by islets and escape via diffusion through the encapsulating alginate. Therefore, the initial O₂ concentration in the replenishment gas mixture was required to be higher than the measured minimum pO₂ values summarized in Table 1. To determine the required initial pO₂, devices loaded with 2,400 IEQ at various densities were implanted in diabetic rats with different initial oxygen concentrations in the oxygen supply container. The gas mixture in each chamber was replenished to its initial level daily. After 24 hours, just before O₂ replenishment, the O₂ concentrations in the oxygen supply container were measured (See, Initial pO₂, Table 2).

Islet Viability and Function after Implantation:

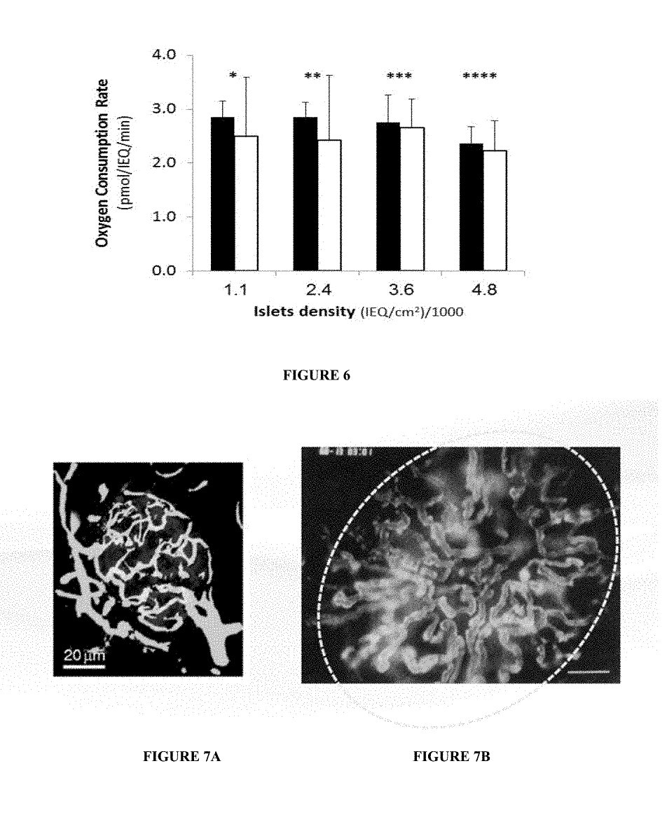

Islets at various densities were immobilized in the device and implanted into diabetic rats. The oxygen supply container was purged with a gas mixture containing the initial required pO₂ (Table 2), and the glycemic parameters in the rats and the OCR of the immobilized islets before implantation and after explantation were measured. The OCR of the islets remained relatively constant with no significant difference between initial and final values (see, e.g., FIG. 6). Normoglycemia was achieved for 90 days (see, e.g., FIG. 5 A), and IVGTT were near normal when tested after 42 days with little or no difference between the normal rats and diabetic rats with implanted devices (see, e.g., FIG. 5 B). The data demonstrates the ability of the device to support functional islets at high densities providing that the initial O₂ concentration in the oxygen supply container is sufficiently high (see, e.g., FIG. 5 B). At high islets density of 4,800 IEQ/cm2 a non-stable glucose levels were obtained (see, e.g., FIG. 5 A), suggesting that this is the maximum density that can be achieve in this setting. In this study all devices were electively removed resulting in the returned of the blood glucose level to the disease state. The minimum implantation period was 90 days. In many rats devices were removed after longer periods of up to 220 days. These data show the device according to some embodiments of the present invention is capable of maintaining the viability and the functionality of the transplanted tissue for at least 90 days.

Table 2: Initial and Final Oxygen Concentrations in the Oxygen Supply Container:

Table 2 summarizes the initial pO₂ and average final pO₂ after 24 hr. The average O₂ concentration in the oxygen supply container after 24 hours at each islet density equaled or exceeded the minimal O₂ level needed, thereby indicating that the initial pO₂ levels used were sufficient to maintain the functionality of the islets.

| | |
|---|---|
| | O₂ partial pressure in the |
| Islet density | Oxygen Supply Container (mmHg) |

| (IEQ/cm³) | Initial pO₂ | Final pO₂ after 24 h |
|---|---|---|
| 48 | 304 | 190 ± 22 |
| 72 | 456 | 280 ± 24 |
| 96 | 570 | 350 ± 45 |

Devices containing islets at various densities were implanted into diabetic rats. The oxygen supply container was daily flushed with 20 ml gas mixtures containing various oxygen concentrations, 40 mmHg CO₂, and balance nitrogen. The pO₂ in the oxygen supply container was measured after 24 h, just prior to flushing the fresh gas mixture. (N=200 different samples for each islet density.)

A native pancreatic islet is well vascularized (FIG. 7A, B), which results in nearly uniform oxygen concentration of 38-40 mmHg throughout the islet. In isolated islets, O₂ must diffuse from the surface into the islet core; therefore, a higher concentration of O₂ must be supplied on the surface of an islet. The subcutaneous (SC) is a site for transplantation; however, the O₂ level in the SC is only about 40 mmHg, which is insufficient to fully support islets above about 100 μm in diameter and will lead to deleterious effects on insulin secretion from human islets that typically average about 150 μm.

FIG. 7 shows pictures of naïve islets, which are highly vascularized, result in about 45 mmHg throughout the islet. FIG. 7 B incorporates a dotted circle indicating the estimated circumference of the islets. The dyed tubes within the broken line are arterioles within the islets supplying the blood to the islet. Isolated islets have a disrupted blood supply and all nutrients and products (e.g. insulin, glucagon) must travel via diffusion. Oxygen is the first molecule to be limited.

The results herein illustrate the relationship between higher density of encapsulated islets and the initial oxygen level required in the oxygen supply container in order to maintain the islets fully viable and functional. Methods to determine the oxygen level needed in the oxygen supply container for a given islet density in a device according to some embodiments of the present invention. This oxygen level would ensure that at the end of a 24 hr period post-replenishment with a gas mixture—just before replenishing the oxygen supply container with a gas mixture containing the initial required pO₂—the minimum pO₂, at the surface of islets nearest the host-device interface (FIG. 8B, 802) would be about 50 mmHg or greater.

In vitro measurements made of the pO₂ profile (See FIGS. 3 and 4) in the transplanted tissue were used to determine the approximate required pO₂ in the gas mixture in different islet density (Table 1).

For example, to support the highest density tested of 4,800 IEQ/cm², a minimum pO₂ of about 305 mmHg is needed. When implanted, an initial oxygen supply container pO₂ of 570 mmHg dropped after 24 hr to about 350 mmHg. Thus, 570 mmHg is sufficient to ensure the functionality of all islets at a density of 4,800 IEQ/cm². One important result of this finding is that the size of a device for implantation in humans can be substantially reduced. Consequently, for example, a dose of 250,000 IEQ could be supported under these conditions in a device having about 50 cm² surface area for supply of O₂ from the oxygen supply container. By mating two devices back to-back with an oxygen supply container between them, the surface area required for islet support could be farther reduced to 25 cm², which is equivalent to a coin-like device with a diameter of less than 6 cm. Such size reduction would make implantation in humans more feasible. It is possible that even higher densities can be supported with further increase in the initial oxygen level in the oxygen supply container, thereby facilitating devices of even smaller size.

Virtually all of the viable islets initially implanted in the device retained their viability throughout the duration of these experiments, even at the highest density of 4,800 IEQ/cm², as demonstrated by maintenance of the OCR until explantation (see FIG. 5A and FIG. 6). Because the pO₂ in the microvasculature is about 40 mm Hg, this result could not have been achieved by any other method that relies upon the local oxygen supply through the bloodstream, even if extensive neovascularization surrounding the implant had been achieved. Furthermore, the devices containing 2,400 IEQ at densities from 1,000 to 4,800 IEQ/cm² implanted into diabetic rats maintained normal fasting blood glucose until elective termination of the experiments after up to 256 days, and no detectable delay in the IVGTT was observed (see FIG. 5 B), demonstrating fast response of the device implant in the subcutis of a rat. Thus, islets immobilized at very high densities in the alginate hydrogel layer survived in the subcutaneously-implantable device for a long period of time without apparent function deterioration.

Typically, clinical islet transplantation requires about 10,000 IEQ/kg BW (about 700,000 islets for a 70 kg patient), usually supplied from two or more cadaveric donors, for most recipients to become insulin independent, whereas only about 10 to 20% of the one million endogenous islets are needed to maintain euglycemia in a normal human. Studies in mice and clinical islet transplantation typically indicate that only about one-third of the islet dose survives the implantation and early engraftment period.

The method presented in this example demonstrates that enhanced in situ supply of exogenous oxygen directly to islets encapsulated at high densities can maintain the viability and function of the islets in their initial state without significant loss over long periods of time. By eliminating the substantial loss of viability and function currently experienced, the limited supply of islets can be used much more efficiently, and human preparations normally discarded because of insufficient numbers of islets can be used fruitfully.

Prevention of islet cell death by ensuring sufficient oxygen supply may provide an additional benefit of enhancing the attainment of true immunoisolation of encapsulated allogeneic or xenogeneic islets, thereby eliminating the need for immunosuppression. Allograft rejection is mediated primarily by the direct pathway, which requires direct contact between donor-derived antigen-presenting cells (APCs) and host-derived T cells. Direct cell contact is prevented by microporous membranes or polymer encapsulation, both of which are used in the device studied here. Consequently, the presence of dying cells in encapsulated islets may generate a large immunogenic stimulus, especially with xenografts, that triggers the indirect pathway and ultimately leads to attack by agents released from activated immune cells that form a florid response around the implant. By maintaining the viability of virtually all islets encapsulated in the device, development of this immunogenic stimulus is prevented, and immunoisolation can be attained.

Table 3 below shows a summary of the device of the present invention:

TABLE 3

| | | Gas | | | Liquid | |
|---|---|---|---|---|---|---|
| | % | mmHg | μM | % | mmHg | μM |
| Min | 30 | 305 | 410 | 6 | 60 | 81 |
| Max | 65 | 494 | 664 | 35 | 360 | 484 |

Table 3 shows conversion of the units of oxygen partial pressure p. For example, at steady-state ambient O₂ partial pressure of 160 mmHg (21% O₂, 1 atm), dissolved O₂ concentration is 215 μM in the medium at 37° C.

FIG. 9 shows embodiments of the device of the present invention, illustrating the device implanted in a rat.

FIGS. 10 A and 10 B show embodiments of the device of the present invention, illustrating rats' islets immobilized within the device prevented oxygen supply after implantation (day 0—day of implantation, FIG. 10 A; 59 days after implantation, FIG. 10 B). FIG. 10A shows implantations without having an oxygen supply; FIG. 10 B shows (via arrow) when the oxygen was replaced with nitrogen, after 59 days of implantation, resulting in return of the glucose blood level to the disease sate. Thus, these data indicate that oxygen must be continuously supplied. Notably, blood glucose is measured to identify how much glucose is being used by the cells i.e., less glucose means that fewer cells are surviving and/or are healthy.

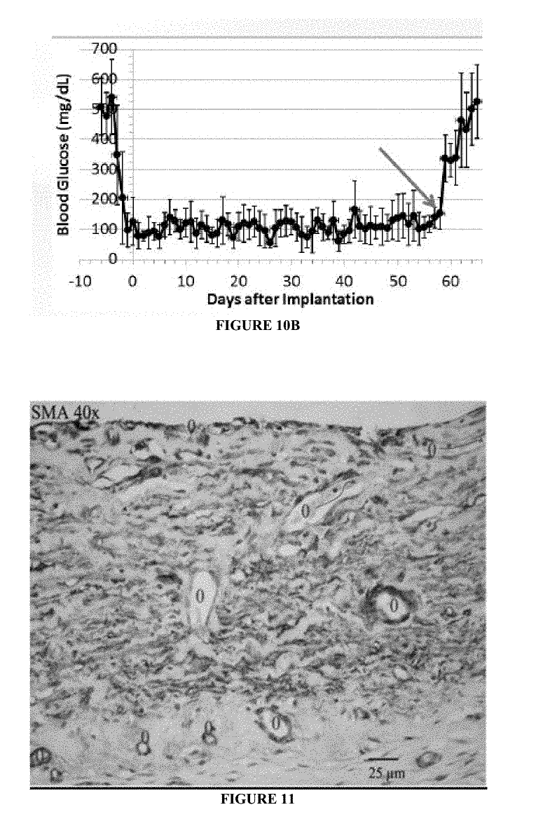

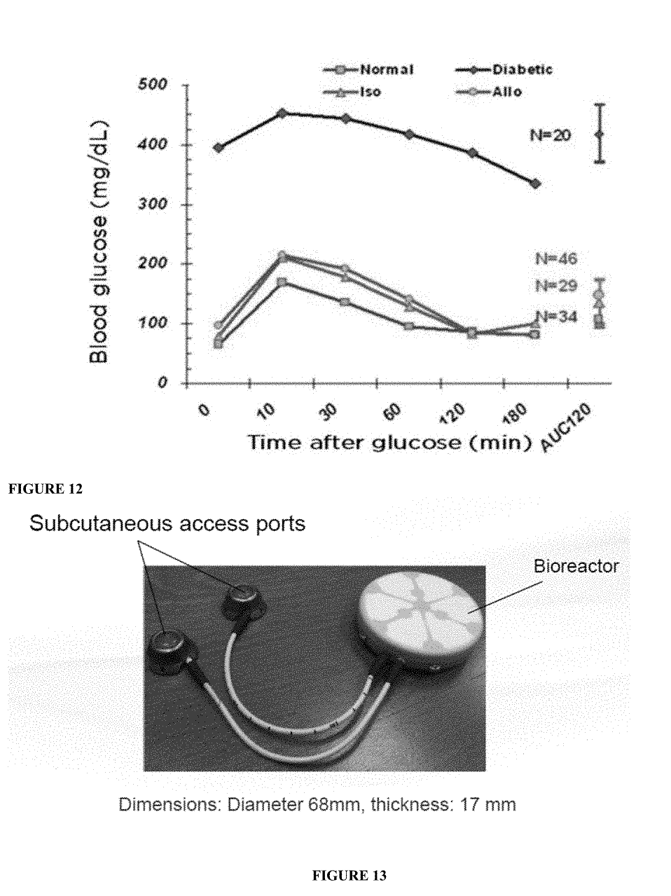

FIGS. 11 and 12 show embodiments of the device of the present invention, where FIG. 11 illustrates a fibrotic pocket surrounding a device explanted from a rat implanted with the device for a period of 140 days. The fibrotic tissue around the device was well vascularized, resulting in about normal IVGTT (intravenous glucose tolerance test), as shown in FIG. 12. The transplanted cells within the device can be isogeneic (triangle) or allogeneic (circle). Similar blood glucose results were obtained from normal, non-diabetic rats (square) and diabetic rats implanted with a device housing either isogeneic or allogeneic transplanted cells. As a negative control, diabetic rats (diamond) without a device implanted had significantly higher blood glucose levels than compared to the non-diabetic rats, or diabetic rats having the device (with allogeneic or isogeneic cells). The diabetic rats had approximately 4 times the amount of blood glucose than the non-diabetic (normal) rats and the rats having the implanted device (with allogeneic or isogeneic cells). These data suggest that the device according to some embodiments of the present invention is capable of functioning in vivo for prolonged periods of time.

### Example 2: Treatment of Diabetic Pigs with a Device According to Some Embodiments of the Present Invention

To support a large animal (e.g. mini-pig of about 10 Kg) a large device was constructed (Actual view in FIG. 13, cross-section view In FIG. 14). Diabetic pigs received devices of the present invention containing initial rat islets dose was about 6,500 IEQ/kg body weight (N=4 mini-pigs). The device was assembled according to the method described in Example 1.

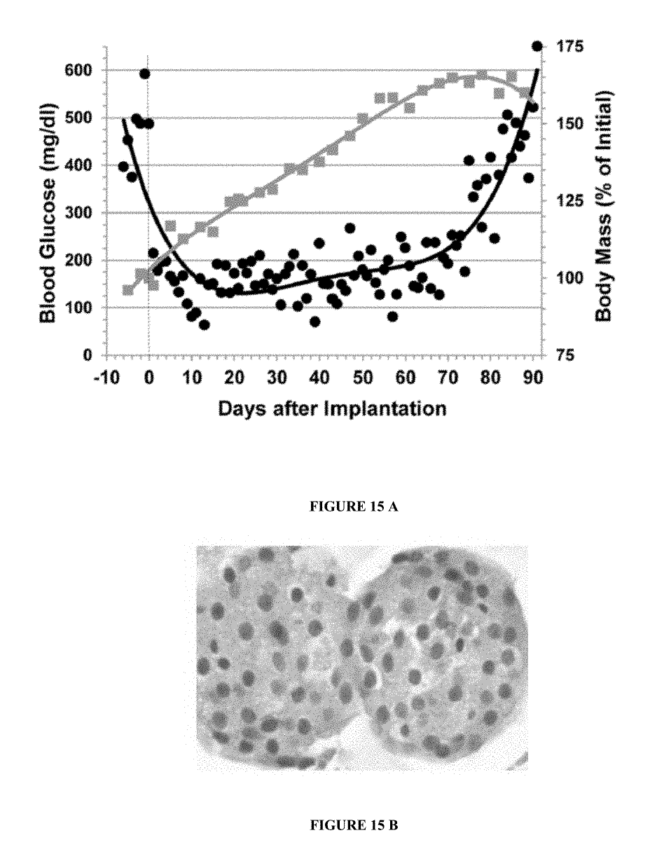

FIG. 15 A shows the average blood glucose levels and weight in diabetic pigs following implantation. The data showed that initially, the blood glucose values were adjusted near normal. Squares indicate body mass (% of initial) and circles represent blood glucose (mg/dl). Pigs gained weight during the implantation period. These data show that that a low dose of rat islets (6,500 IEQ/kg body weight) implanted within the device can cure STZ mini-pig. Surprisingly, the device could support the pigs up to 80 days post-implantation. However, after the 80 days, the implanted devices were no longer able to maintain normal blood glucose levels, possibly because the pig's body weight was too large for the dose of islets implanted. After 89 days, the devices were removed, the islets retrieved, and stained for insulin. Referring to FIG. 15 B, insulin staining was observed in the retrieved islets.

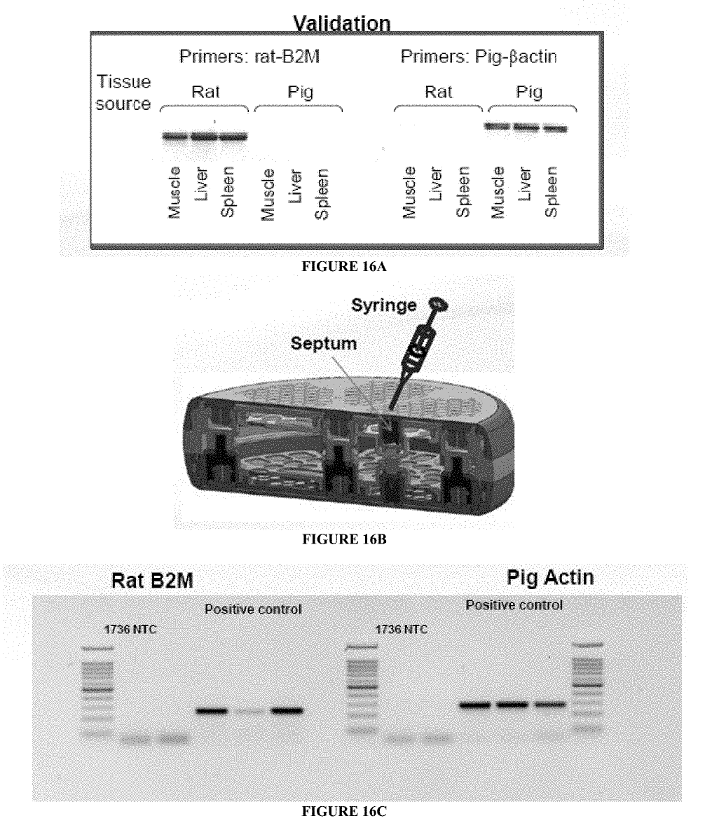

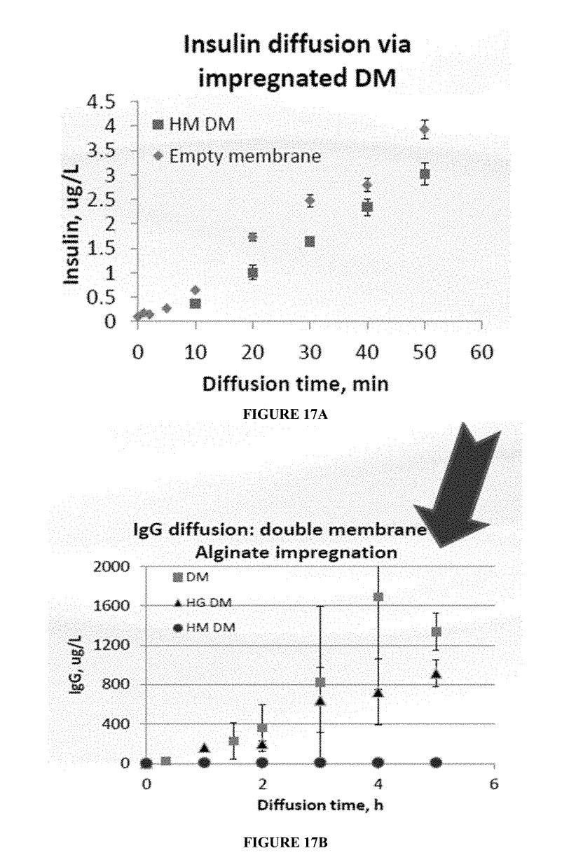

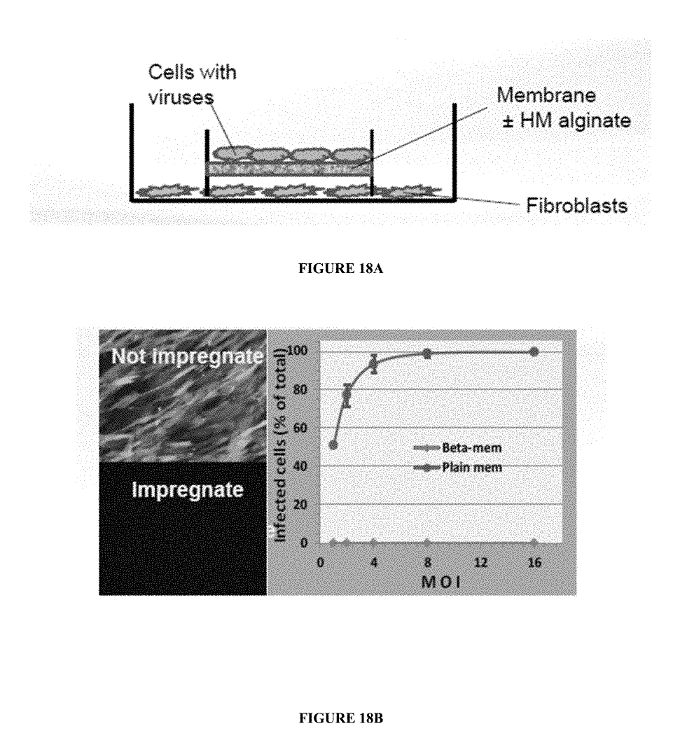

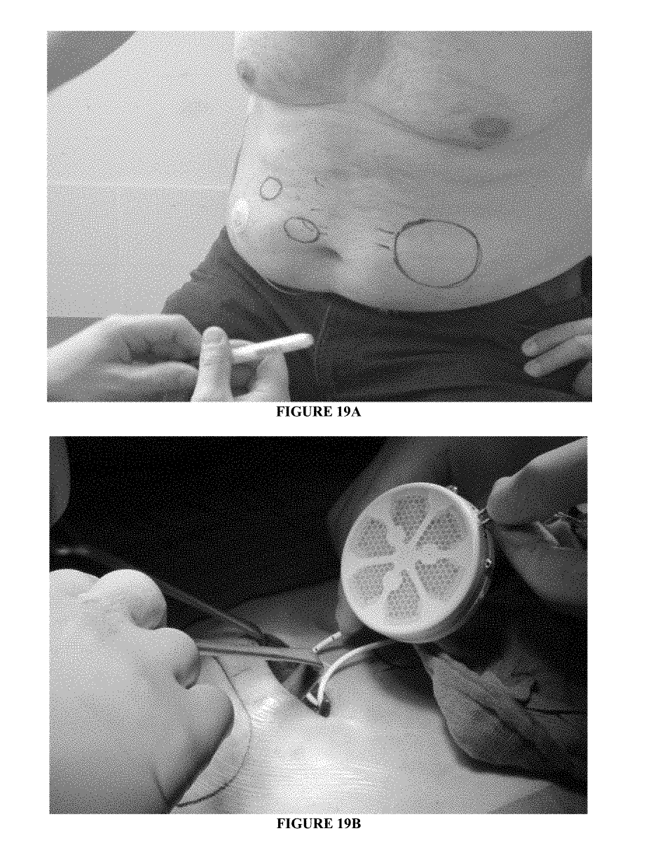

Referring to FIG. 16, it can be seen that no rat or pig cells were found in these samples, indicating that the membrane of the device was shown to restrict the transplanted cells from entering the environment outside of the device. Furthermore, no DNA from the rat or pig was found in the device. Accordingly, these experiments (FIG. 16 C) show that the device is protected from the immune system of the implanted mammal.

### Example 3: Permeability of the Device According to Some Embodiments of the Present Invention to IgG and Insulin

FIGS. 17 A and 17 B show graphs of molecule transfer via the membrane of embodiments of the device of the present invention. The results of FIG. 17 A show insulin diffusion through the membrane (Teflon, 0.4 microns). Although the membrane was blocked using HM DM, insulin was still able to pass through the membrane. These data show that transfer rate of insulin was not affected by the membrane and the transfer rate of IgG is significantly hindered. FIG. 17 B further shows that insulin is able to cross the impregnated membrane (which includes islets/membrane/alginate) of the device, while blocking IgG antibodies (circles). An unimpregnated membrane (square—DM), i.e., without alginate, the IgG will cross through the membrane.

FIGS. 18A and 18 B show embodiments of the device of the present invention, showing virus protection. Cells with different virus loads were seeded on top of impregnate Biopore membrane and the existence of virus in the fibroblast below was tested. The impregnated alginate completely stopped the virus penetration. FIG. 18 A shows a cartoon of a membrane impregnated with alginate blocking IgG and a ˜70K virus from crossing the membrane. FIG. 18 B shows that the virus was able to migrate across an unimpregnated membrane, while an impregnated membrane was devoid of virus—thus, no migration.

### Example: Treatment of Diabetic Humans with a Device According to Some Embodiments of the Present Invention

Preclinical results in two animal models proved the ability of the device to: (1) support oxygen requirements of the donor; (2) protect isogenic, allogenic, and xenogenic implanted cells from the host immune system; (3) achieve near-normal glucose control in diabetic animals; and (4) achieve glucose pharmacokinetics pattern in diabetic animals (rats and pigs) similar to a healthy animal pattern.

### Clinical Study

The subject was a 63 years old male, and was a diagnosed type I diabetic (T1D) since 1957. He did not have any relevant complications, and had an acceptable glycemic control under CSII. The trial design was as follows: the macroencapsulated human islets (with marginal mass of 2,100 IEQ/kg BW) were subcutaneously transplanted. There was no immunosuppression provided. The primary endpoint was to assess safety and feasibility (including oxygen refueling), and the secondary endpoint was to study metabolic control (e.g., monitoring HbA1c), determining the insulin requirement, and assessing a positive C-peptide.

FIG. 19 A is a picture of the subject pre-surgery, with marks of the device and ports to be implanted. FIG. 19B is a picture of the device being implanted. FIG. 19C is a picture of the ports being implanted. FIG. 19D is a picture of the device subcutaneously implanted.)

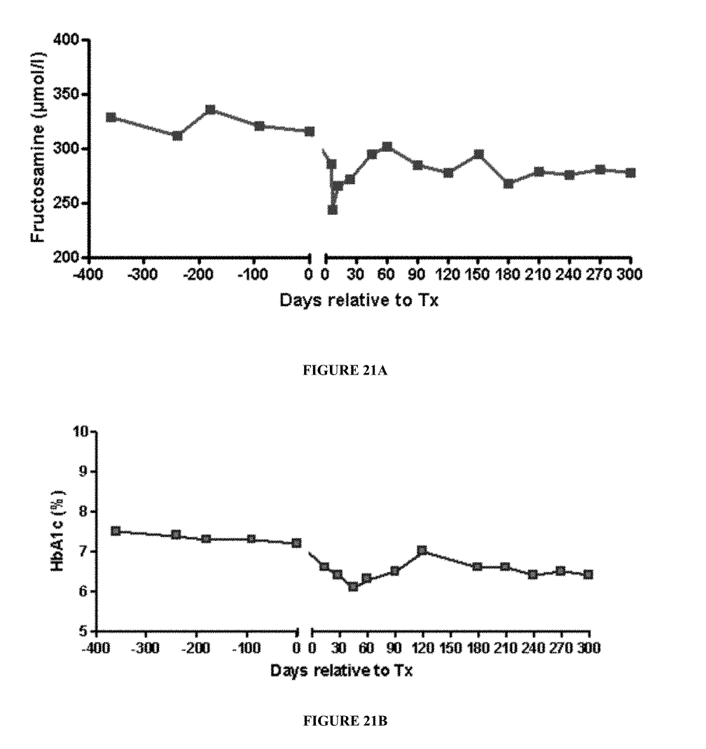

FIG. 20 A shows that a patient using a minimum amount of insulin (prior to implantation of the device) had large deviations in blood glucose levels over a period of about 24 hours. However, this same subject, after implantation of the device, had a more level average of blood glucose over a period of about 24 hours (see FIG. 20 B). The measurements obtained in FIG. 20B were obtained 1 month after the subject had the surgery implanting the device.

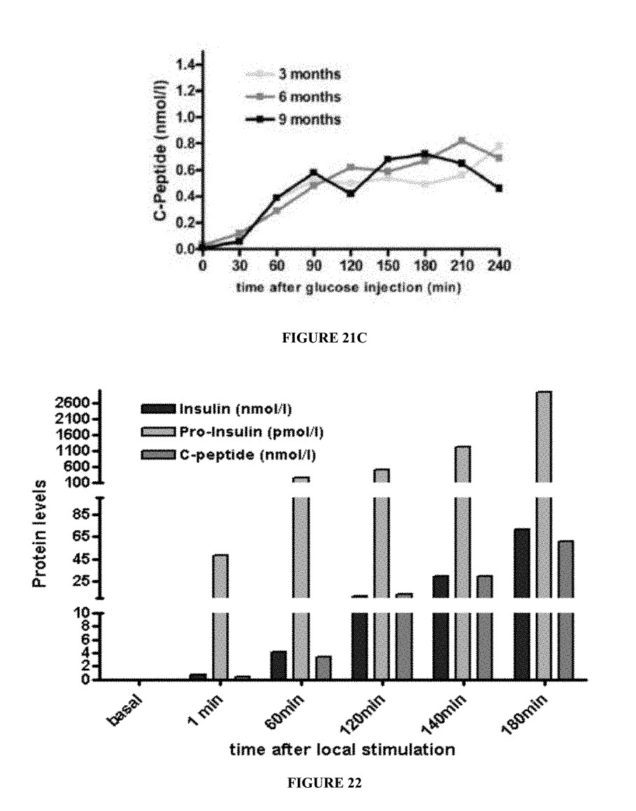

FIGS. 21A-C show embodiments of the device of the present invention, illustrating via graph the metabolic findings of the clinical trial. FIG. 21 A shows the metabolic results measured over days post treatment of a single patient, illustrating that the levels of fructosamine were substantially linear after implantation of the device, measuring between about 250 and 300 μmol/L. FIG. 21 B shows that oxygen can be injected to sustain 160,000 functional islets (4,500 IEQ/cm²) in a subject—and the device was partially damaged, thus only one half of the islets proved functional. FIG. 21 B shows that the implanted device decreased HbA1c (%) by between 1-2%. FIG. 21 C shows that the results of injecting glucose locally. A secretion of C-peptide was demonstrated after 3, 6, or 9 months post-implantation, testing c-peptide concentrations between 30-240 minutes after glucose injection. Each of the 3, 6, and 9 month samples showed similar results and progression of c-peptide increase over time (e.g., after 60 or 90 minutes, the 3, 6, and 9 month samples deviated less than 0.5 nmol/L. This data indicates stable functionality of the device for a period of 9 months.

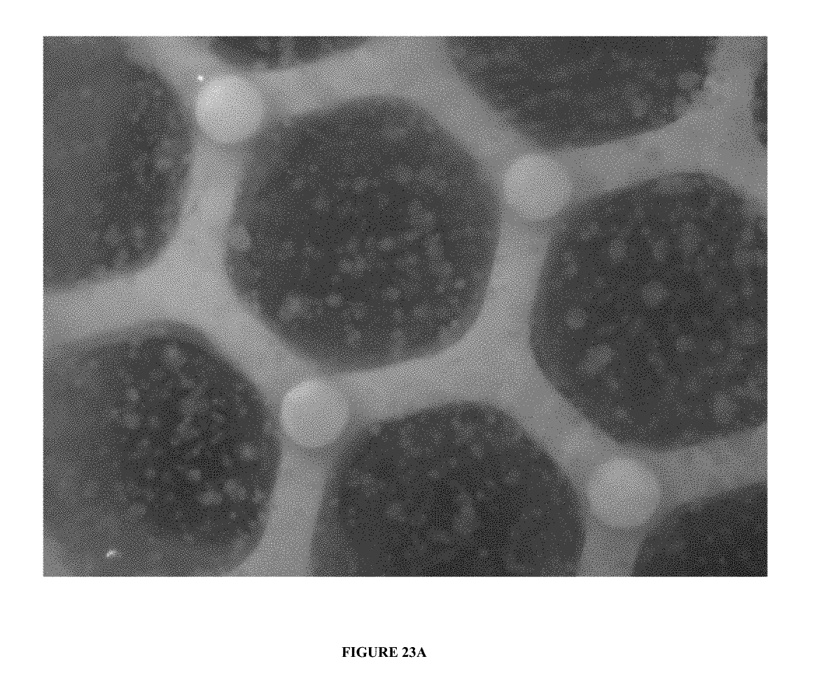

The subject was injected locally around the device with high glucose (15 mM) solution and the local hormone concentrations of insulin, pro-insulin, and c-peptide were evaluated over a period of 180 minutes. The results in FIG. 22 show viable and functional islet grafts. The functional device was re-located to a potentially favorable site, without inappropriate invasiveness.

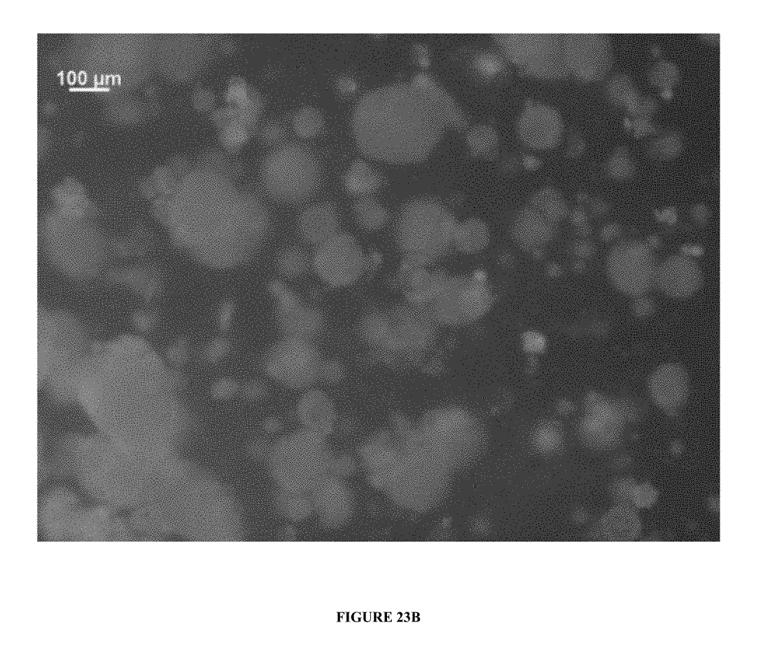

FIGS. 23A and 23 B show two microscopy pictures showing the embodiments of the device of the present invention after retrieval from the clinical study. FIG. 23 A is bright field microscopy showing intact islet structures after being removed from a device previously implanted 10 months earlier in a subject. FIG. 23B shows dithizone staining for insulin which is homogenous and intense in the bottom-side of the islet-containing hydrogel layer, heterogeneous and diminished staining in the upper-side of the islet-containing hydrogel layer (data not shown). Therefore, FIG. 23 B shows that the transplanted cells are active after 10 months of implantation in a patient without immune-suppression.

FIGS. 24A and 24 B show graphs of embodiments of the device of the present invention. The graphs were generated following retrieval of the device from the human subject following 10 months implantation and the device was incubated in 20 mM glucose solution and protein levels were tested. Alginate layers containing the transplanted tissue were removed from the device and HG-alginate content was 5.5 micrograms/mL compared with 4.23±0.86 micrograms/mL prior to implantation. HM-alginate content in the layers containing the transplanted tissue was 24±4 and 18±5 micrograms/mL, respectively, compared with 25±2.6 before implantation. Therefore, the content of HM and HG alginates remain as before implantation, suggesting a stable gel system. The device was removed from a subject after being implanted in the subject for 10 months and the transplanted cells of the device were tested and were found to be functioning normally.

Therefore, the first human trial of macroencapsulated allogeneic islet transplantation (10 months follow up) demonstrated: feasibility and safety of the implantation and the oxygen refueling procedure, biocompatibility of the device, survival of allogeneic islets without immunosuppression due to the continuous supply of oxygen, and preservation of glucose responsibility. Islet graft function was directly proven by detection of human C-peptide following stimulation (despite a very marginal islet mass) in an initially C-peptide negative patient.

### Example 5: Xenogeneic Implantation: Treatment of Diabetic Rats with a Device Containing Human Islets According to Some Embodiments of the Present Invention

Human islets were purchased from Prodo (CA). Upon arrival about 500 islets were located in 90 mm petri dish and cultured with 10 ml of RMPI/CMRL (50/50%) supplemented with 10% calf serum (Bet-Hemek, Israel).

A dose of 2,400±200 human IEQ was collected by 5-min sedimentation. The pellet was gently mixed with 2.2% (w/v) ultrapure high-guluronic acid (68%) alginate (Pronova UPMVG, Novamatrix; Sandvika, Norway). The mixture was placed in a device according to some embodiments of the present invention according to the method described in Example 1.

Rats were anesthetized by intraperitoneal injection of 90 mg/Kg Ketamine and 10 mg/Kg Xylazine followed by isoflurane inhalation. A 3-cm incision was made for the device on the dorsal skin, and muscles were separated from the hypodermis. A second incision was made in the skin between the shoulder blades, and two channels connecting this site with the device implantation site were created by traversing 3-mm wide stainless steel needles under the skin. The device was inserted under the dorsal skin incision with the islet module facing the fascia, and the gas chamber ports were connected to the remote subcutaneous access ports. The skin was sutured and fixed with a tissue adhesive (Histoacryl, Tufflingen, Germany).

Every 24 h the animal was sedated with isoflurane inhalation. A 27G needle was inserted into each of the two implanted access ports, and the gas chamber was purged with 20 ml (about 6.7 chamber volumes) of gas mixture containing 456 mmHg of O2, 40 mmHg CO₂, and balance N₂ (Maxima, Israel). Final total pressure in the gas chamber was equal to ambient atmospheric pressure.

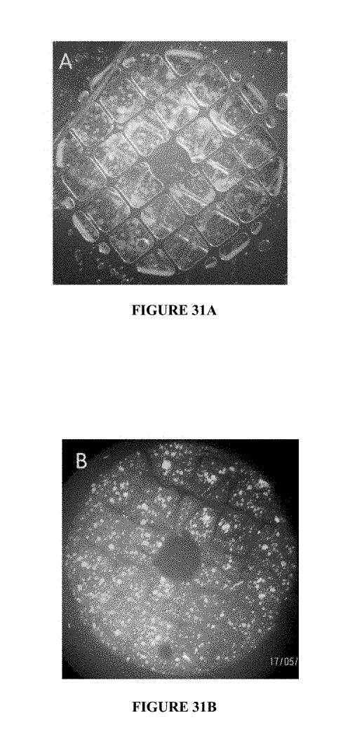

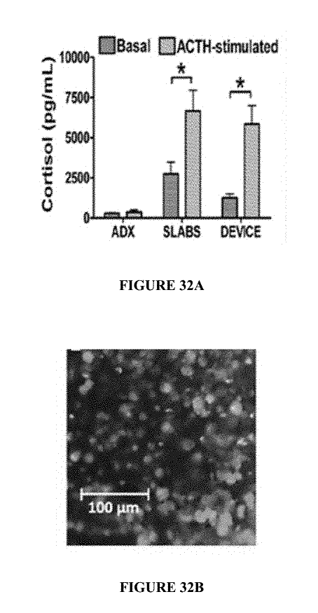

FIGS. 31A and 31B show human islets in a device according to some embodiments of the present invention. FIG. 31 A: A micrograph of human islets in a device according to some embodiments of the present invention prior to implantation in a rat. FIG. 31 B: A micrograph of human islets in a device according to some embodiments of the present invention in a device removed from a rat after being implanted for one month. These data demonstrate that a device according to some embodiments of the present invention is capable of maintaining the viability of human islets.

### Example 6: Implantation of a Device According to Some Embodiments of the Present Invention Containing Adrenal Cells into Adrenalectomized Rats

BAC (Bovine adrenal cells) were isolated from bovine adrenals of freshly slaughtered 1-3-y-old cattle by collagenase digestion, according to the methods described in Haidan A, et al., 1998; Vukicevic V, et al., 2012; Chung K F, et al., 2009).

Pelleted BACs were gently mixed with 3.5% (wt/vol) sterile high guluronic acid (HG) alginate, dissolved in Custodiol-HTK solution (H.S. Pharma). The alginate—cell mixture was then placed either on a glass (for slabs) or spread in the cell compartment of the chamber device. Alginate was cross-linked by applying flat Sintered glass (Pyrex), saturated with 70 mM strontium chloride plus 20 mM Hepes. The thickness of alginate/cell slab was about 550 μm.

Female RNU (8 wk old) and Wistar rats (200 g) were obtained from Charles River Laboratory. Bilateral adrenalectomies were performed simultaneously with the cell transplantation procedure. For naked cell transplantation, 5×10⁶ of bovine adrenocortical cells were infused into a pouch formed under the capsule of each kidney. For encapsulated cell transplantation, two identical slabs were implanted, one underneath each kidney capsule. For i.p. transplantation in adrenalectomized Wistar rats, two slabs, containing 5×10⁶ cells each, were carefully placed into the retroperitoneal space. Macrochambers were placed under the dorsal skin. 20 ml of oxygen-enriched gas mixture (60% oxygen, 35% nitrogen, 5% CO₂) was used for daily exchange of the gas.

FIG. 32 A shows basal and ACTH-stimulated plasma cortisol levels in adrenalectomized rats (ADX), adrenalectomized rats implanted with a device according to some embodiments of the present invention containing bovine adrenal cells (DEVICE), and adrenalectomized rats implanted with alginate hydrogels containing bovine adrenal cells (SLABS). FIG. 32 B shows the viability of bovine adrenal cells in a device according to some embodiments of the present invention. Data was obtained following 20 days of implantation. These data demonstrate that a device according to some embodiments of the present invention is capable of maintaining the viability of bovine adrenal cells.

### Example 7: Treatment of Diabetic Rats with a Device According to Some Embodiments of the Present Invention Containing Human Embryonic Stem Cell-Derived Insulin Producing Cells

Pluripotent maintenance of the human embryonic stem cell line WA01 (H1) was accomplished through co-culture with irradiated mouse embryonic feeders. Differentiation of the pluripotent cells occurred by passage onto growth-factor depleted Matrigel (BD Biosciences 354230) followed by 3 days of growth in MEF-conditioned medium before initiating the differentiation protocol.

A stage-wise description of the differentiation protocols used is disclosed in U.S. Pat. No. 8,338,170 and is briefly described with specific additive concentrations. Stage 1 consisted of a 3-day incubation in RPMI containing 100 ng/ml Activin A (Peprotech 120-14), 8 ng/ml bFGF (Life Technologies 13256029) and 20 ng/ml Wnt3a (R&D 5036-WN/CF). Wnt3a was only applied on the first day of stage 1, aiding the formation of definitive endodermal cells. Stage 2 consisted of an 8-day incubation in DMEM/F12 containing 2 μM retinoic acid (Sigma Aldrich R2625), 100 ng/ml Noggin (R&D 3344-NG), 250 nM cyclopamine (Calbiochem 239804), 100 ng/ml Fgf10 (Peprotech 100-26) and 1% Hyclone defined FBS (Thermo Scientific SH300700,02) for the first four days and 1% B27 (Life Technologies 08-00855A) for the final four days. Stage 3 consisted of a 3-day incubation in DMEM/F12 containing 2 μM retinoic acid, 100 ng/ml Noggin, 250 nM cyclopamine, 20 ng/ml Wnt3a, 50 ng/ml Activin A and 1% B27. Stage 4 consisted of a 12 day incubation in DMEM/F12 with 12 mM Glucose supplemented with 50 μM DAPT (Sigma Aldrich D5942), 0.5 μM 1.25 (OH)2 Vitamin D3 (EMD Chemical 679101), 1 μM ALK5 inhibitor (A-83-01, EMD Chemical 616452), 1 mM Sodium Propionate (Sigma Aldrich P1880) and 50 μM 8-Br-cAMP (Sigma Aldrich B7880).

The human islets used as controls in this study were obtained from the Islet Isolation Program at U. Illinois Chicago (Dr. J. Oberholzer). Human islets were maintained in complete islet medium composed of Final Wash/Culture Medium (Cellgro 99-785-CV, Corning, Va.) supplemented with 2.5% Human Serum Albumin (Grifols NDC 68516-5216-2, CA), 0.244% Sodium Carbonate (Hospira 0409-6625-02, CA), 10 mM HEPES (Mediatech-Cellgro 25-060, VA), Ciprofloxacin (Hospira 0409-4778-86, CA) and 0.2% Insulin-Transferrin-Selenium (Invitrogen 41400-045). The medium was replenished every other day.

Stage 4 cells were detached from the flask culture by 5 minutes incubation with collagenase followed by gentle pipetting. Flasks were then pooled and an aliquot was treated with trypsin to estimate the cell number. Cultures were then grown overnight in suspension as cellular aggregates.

3×10⁶ hESC cells (n=3), or 3,000 human islets were mixed with 2.5% HG (G=0.68) alginate loaded in each βAir device. Devices incubated for 16 minutes in a Strontium solution (70 mM SrCl2, 12.5 mM NaCl, 20 mM Hepes pH 7.4) to establish cross-linking of the alginate. Excess Strontium solution was washed off the alginate/cell slab in the complete islet medium. The impregnated Biopore membrane was glued to the body of the device by Silicone glue (Millipore SLGSM33SS) and an O-Ring was mounted on the membrane. Devices were implanted.

Lewis rats (8 w gestational age at implantation, ranging between 190-216 grams) were maintained on a high fat diet to assist in weight gain. Devices were refueled daily with a gas mixture composed of 55% nitrogen, 40% oxygen and 5% carbon dioxide (Praxair special order). Briefly, rats were anesthetized using an isoflurane chamber. The skin covering the refueling ports was washed with ethanol and a 27 gauge needle (BD 305109) was inserted into the each port. A filtered (Millipore SLFG025LS) syringe (BD 302832) containing 20 ml gas mixture was affixed to one of the needles (the side that the gas mixture was injected into was changed daily) while the other served as an exhaust for the displaced used gas present in the device. For collection of blood, rats were bled through the tail vein bi-weekly. Blood samples were pelleted and the supernatant was subjected to ELISA analysis to determine fed hC-peptide levels in circulation.

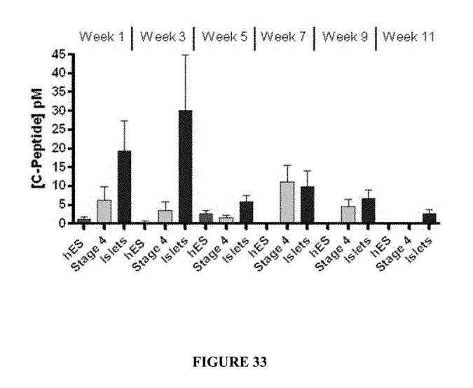

The devices were implanted in rats and the levels of human C-Peptide in the blood were followed. Stage 4 cells showed persistent C-peptide secretion up to week 9 after implantation (Light brown columns, see FIG. 33).

These data demonstrate that a device according to some embodiments of the present invention is capable of maintaining the viability of insulin producing cells derived from human embryonic stem cells implanted within the device in rat xenogeneic system.

Publications cited throughout this document are hereby incorporated by reference in their entirety. Although the various aspects of the presently disclosed embodiments have been illustrated above by reference to examples and preferred embodiments, it will be appreciated that the scope of the presently disclosed embodiments are defined not by the foregoing description but by the following claims properly construed under principles of patent law.

In addition, citation or identification of any reference in this application shall not be construed as an admission that such reference is available as prior art to the presently disclosed embodiments. To the extent that section headings are used, they should not be construed as necessarily limiting.

What is claimed is:

1. A device containing transplanted cells, comprising:
a housing, having a chamber, defined by a top, a bottom surface, and sides, configured for insertion into a body of a subject, comprising:
a. an oxygen supply container, having a chamber, defined by a top surface, a bottom surface, and sides, disposed within the chamber of the housing, wherein the top surface and the bottom surface of the oxygen supply container comprise at least one gas-permeable membrane,
b. at least one hydrogel layer, having an inner surface, and an outer surface, wherein the inner surface of the at least one hydrogel layer contacts at least one surface selected from the group consisting of: the top surface of the oxygen supply container, and the bottom surface of the oxygen supply container, wherein the at least one hydrogel layer contains the transplanted cells;
c. at least one port, configured to deliver oxygen to the oxygen supply container, wherein the at least one port is fluidly connected to the chamber of the oxygen supply container; and
d. at least one access port, configured to receive an exogenous supply of gas, fluidly connected to the at least one port,
wherein the device is configured to promote the survival and/or function of the transplanted cells;
wherein the oxygen supply container is further configured to supply oxygen to provide a minimum pO2 of between a value of 20-600 mm Hg for at least 24 hours, and
wherein the oxygen supply container is further configured to be periodically replenished with oxygen.

2. The device of claim 1, wherein the at least one hydrogel layer comprises guluronic acid alginate.

3. The device of claim 1, wherein the transplanted cells are selected from the group consisting of islets of Langerhans, stem cells, adrenal cells, insulin secreting cells, beta cells, stem cell-derived insulin producing cells, stem cell-derived beta cells, stem cell-derived alpha cells and genetically modified cells.

4. The device of claim 1, wherein the transplanted cells are human.

5. The device of claim 1, wherein the transplanted cells are selected from the group consisting of allogeneic cells, xenogeneic cells, isogeneic cells, and autologous cells.

6. The device of claim 1, wherein the device protects the transplanted cells from the subject's immune system.

7. The device of claim 1, wherein the outer surface of the at least one hydrogel layer comprises an immune protection membrane.

8. The device of claim 7, wherein the immune protection membrane comprises porous polytetrafluoroethylene or collagen.

9. The device of claim 1, wherein the device is implanted into the body of the subject at a location selected from the group consisting of: a subcutaneous location, an intramuscular location, an intraperitoneal location, a pre-peritoneal location, and an omental location.

10. The device of claim 1, wherein the oxygen delivered to the chamber of the oxygen supply container has a concentration between 21% and 95%.

11. The device of claim 1, wherein the oxygen is delivered to the chamber of the oxygen supply container at an initial partial pressure of between 200 and 950 mmHg.

12. The device of claim 1, wherein the transplanted cells contained within the at least hydrogel layer has a density of a value between 1,000,000 cells/cm² and 100,000,000 cells/cm².

13. The device of claim 1, wherein the transplanted cells contained within the at least one hydrogel layer has a density of a value between 1,000 IEQ/cm² and 15,000 IEQ/cm².

14. The device of claim 1, wherein the least hydrogel layer has a uniform thickness between 100 and 800 micrometers.

15. The device of claim 1, wherein the at least one access port is implanted remotely from the apparatus.

16. The device of claim 15, wherein the at least one access port is implanted into the body of the subject at a location selected from the group consisting of: a subcutaneous location, an intramuscular location, an intraperitoneal location, a pre-peritoneal location, and an omental location.

17. The device of claim 1, wherein oxygen passes from the chamber of the oxygen supply container to the transplanted cells contained within the hydrogel layer through the at least one gas-permeable membrane of the oxygen supply container.
